(19) United States
(12) Patent Application Publication
(10) Pub. No.: US 2026/0215791 A1
(43) Pub. Date: Jul. 30, 2026

(54) RIGHT ANGLE DRILL, INSTRUMENTATION, SYSTEM AND METHODS OF USE AND ASSEMBLY

(71) Applicant: Paragon 28, Inc., Englewood, CO (US)

(72) Inventors: Dylan PAPROSKI, Littleton, CO (US); Daniel J. LEE, Denver, CO (US); Sven BLACHE, Wellendingen (DE); Marc MUNDHENKE, Rielasingen-Worblingen (DE)

(73) Assignee: Paragon 28, Inc., Englewood, CO (US)

(21) Appl. No.: 19/573,779

(22) Filed: Mar. 20, 2026

Related U.S. Application Data
(63) Continuation of application No. PCT/US2024/047766, filed on Sep. 20, 2024.
(60) Provisional application No. 63/584,173, filed on Sep. 20, 2023.

(51) Int. Cl. A61B 17/16; A61B 17/17; A61B 17/00; A61B 17/56

(52) U.S. Cl. CPC A61B 17/1682; A61B 17/1775; A61B 2017/00477; A61B 2017/564

(57) ABSTRACT

Right angle drills, instruments, systems and methods for arthroplasty procedures of the ankle joint. The instruments include a coupling portion, an alignment mechanism coupled to the coupling portion, and a driving member coupled to the alignment mechanism, wherein the drill bit is positioned perpendicular to a housing of the driving members.

### CROSS REFERENCE TO RELATED APPLICATION

This application is a continuation of International Application No. PCT/US2024/047766 filed September 20, 2024 and entitled Right Angle Drill, Instrumentation, System and Methods of Use and Assembly, which claims priority benefit under 35 U.S.C. §119(e) of U.S. provisional application No. 63/584,173 filed September 20, 2023, which are incorporated herein by reference in their entirety.

### TECHNICAL FIELD

The present disclosure relates generally to general, podiatric, and orthopaedic surgery related to joint deformities and injuries. More specifically, but not exclusively, the present disclosure relates to right angle drills, instruments, systems and methods for arthroplasty procedures of the ankle joint.

### BACKGROUND OF THE INVENTION

Many currently available instruments, systems, and surgical methods for procedures involving the foot and/or ankle do not completely address the needs of patients. Additionally, many currently available instruments, systems, and surgical methods for incorporation in procedures involving the foot and/or ankle, for example, ankle arthroplasty procedures, fail to account for properties of joint anatomy and associated mechanical and kinematic movement patterns and/or capabilities.

Thus, it is an object of the present disclosure to overcome one or more of the above-described drawbacks and/or disadvantages of the currently available systems.

### SUMMARY OF THE INVENTION

The present disclosure is directed toward instruments, systems and methods for procedures involving the foot and/or ankle.

In one aspect of the present disclosure provided herein, is an instrument. The instrument includes a coupling portion, an alignment mechanism coupled to the coupling portion, and a driving member coupled to the alignment mechanism, wherein the drill bit is positioned perpendicular to a housing of the driving members.

In another aspect of the present disclosure provided herein is a surgical method for ankle arthroplasty procedures.

These and other objects, features, and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 is a perspective view of a first instrument system, in accordance with an aspect of the present disclosure;

FIG. 2 is a perspective view of a second instrument system, in accordance with an aspect of the present disclosure;

FIG. 3 is a first perspective view of the instrument of FIG. 2, in accordance with an aspect of the present disclosure;

FIG. 4 is a second perspective view of the instrument of FIG. 3, in accordance with an aspect of the present disclosure;

FIG. 5 is a third perspective view of the instrument of FIG. 3, in accordance with an aspect of the present disclosure;

FIG. 6 is a fourth perspective view of the instrument of FIG. 3, in accordance with an aspect of the present disclosure;

FIG. 7 is a fifth perspective view of the instrument of FIG. 3, in accordance with an aspect of the present disclosure;

FIG. 8 is a first side view of the instrument of FIG. 3, in accordance with an aspect of the present disclosure;

FIG. 9 is a second side view of the instrument of FIG. 3, in accordance with an aspect of the present disclosure;

FIG. 10 is a first end view of the instrument of FIG. 3, in accordance with an aspect of the present disclosure;

FIG. 11 is a second end view of the instrument of FIG. 3, in accordance with an aspect of the present disclosure;

FIG. 12 is a top view of the instrument of FIG. 3, in accordance with an aspect of the present disclosure;

FIG. 13 is a bottom view of the instrument of FIG. 3, in accordance with an aspect of the present disclosure;

FIG. 14 is an exploded, first perspective view of the instrument of FIG. 3, in accordance with an aspect of the present disclosure;

FIG. 15 is an exploded, second perspective view of the instrument of FIG. 3, in accordance with an aspect of the present disclosure;

FIG. 16 is an exploded, first perspective view of the coupling portion of the instrument of FIG. 3, in accordance with an aspect of the present disclosure;

FIG. 17 is an exploded, second perspective view of the coupling portion of FIG. 16, in accordance with an aspect of the present disclosure;

FIG. 18 is an exploded, top view of the coupling portion of FIG. 16, in accordance with an aspect of the present disclosure;

FIG. 19 is an exploded, bottom view of the coupling portion of FIG. 16, in accordance with an aspect of the present disclosure;

FIG. 20 is an exploded, first side view of the coupling portion of FIG. 16, in accordance with an aspect of the present disclosure;

FIG. 21 is an exploded, second side view of the coupling portion of FIG. 16, in accordance with an aspect of the present disclosure;

FIG. 22 is an exploded, first end view of the coupling portion of FIG. 16, in accordance with an aspect of the present disclosure;

FIG. 23 is an exploded, second end view of the coupling portion of FIG. 16, in accordance with an aspect of the present disclosure;

FIG. 24 is an exploded, first perspective view of the alignment assembly of the instrument of FIG. 3, in accordance with an aspect of the present disclosure;

FIG. 25 is an exploded, second perspective view of the alignment assembly of FIG. 24, in accordance with an aspect of the present disclosure;

FIG. 26 is an exploded, first end view of the alignment assembly of FIG. 24, in accordance with an aspect of the present disclosure;

FIG. 27 is an exploded, second end view of the alignment assembly of FIG. 24, in accordance with an aspect of the present disclosure;

FIG. 28 is an exploded, first side view of the alignment assembly of FIG. 24, in accordance with an aspect of the present disclosure;

FIG. 29 is an exploded, second side view of the alignment assembly of FIG. 24, in accordance with an aspect of the present disclosure;

FIG. 30 is an exploded, top view of the alignment assembly of FIG. 24, in accordance with an aspect of the present disclosure;

FIG. 31 is an exploded, bottom view of the alignment assembly of FIG. 24, in accordance with an aspect of the present disclosure;

FIG. 32 is an exploded, first perspective view of the driving member of the instrument of FIG. 3, in accordance with an aspect of the present disclosure;

FIG. 33 is an exploded, second perspective view of the driving member of FIG. 32, in accordance with an aspect of the present disclosure;

FIG. 34 is an exploded, first side view of the driving member of FIG. 32, in accordance with an aspect of the present disclosure;

FIG. 35 is an exploded, second side view of the driving member of FIG. 32, in accordance with an aspect of the present disclosure;

FIG. 36 is an exploded, first end view of the driving member of FIG. 32, in accordance with an aspect of the present disclosure;

FIG. 37 is an exploded, second end view of the driving member of FIG. 32, in accordance with an aspect of the present disclosure;

FIG. 38 is an exploded, top view of the driving member of FIG. 32, in accordance with an aspect of the present disclosure;

FIG. 39 is an exploded, bottom view of the driving member of FIG. 32, in accordance with an aspect of the present disclosure;

FIG. 40 is a first perspective view of a portion of the instrument of FIG. 2 with transparent portions, in accordance with an aspect of the present disclosure;

FIG. 41 is a second perspective view of the portion of the instrument of FIG. 40, in accordance with an aspect of the present disclosure;

FIG. 42 is a first perspective view of a portion of the instrument of FIG. 2, in accordance with an aspect of the present disclosure;

FIG. 43 is a second perspective view of the portion of the instrument of FIG. 42, in accordance with an aspect of the present disclosure;

FIG. 44 is a first perspective view of a portion of the instrument of FIG. 1 with transparent portions, in accordance with an aspect of the present disclosure;

FIG. 45 is a second perspective view of the portion of the instrument of FIG. 44, in accordance with an aspect of the present disclosure;

FIG. 46 is a first perspective view of a portion of the instrument of FIG. 1, in accordance with an aspect of the present disclosure; and

FIG. 47 is a second perspective view of the portion of the instrument of FIG. 46, in accordance with an aspect of the present disclosure.

### DETAILED DESCRIPTION FOR CARRYING OUT THE INVENTION

Generally stated, disclosed herein are right angle drills, instruments, and systems for arthroplasty procedures of the ankle joint. Further, methods for arthroplasty procedures of the ankle joint are discussed.

In this detailed description and the following claims, the words proximal, distal, anterior or plantar, posterior or dorsal, medial, lateral, superior, and inferior are defined by their standard usage for indicating a particular part or portion of a bone, instrument, or implant according to the relative disposition of the natural bone or directional terms of reference. For example, “proximal” means the portion of an instrument nearest the torso, while “distal” indicates the portion of the instrument farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers to the bottom of the foot.

Similarly, positions or directions may be used herein with reference to anatomical structures or surfaces. For example, as the current instrumentation, system, and methods are described herein with reference to use with the bones of the ankle, the bones of the foot, ankle and lower leg may be used to describe the surfaces, positions, directions or orientations of the instrumentation, system, and methods. Further, the instrumentation, system, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the instrumentation, system, and methods, and the aspects, components, features and the like thereof, described and/or illustrated herein may be changed, varied, modified, reconfigured or otherwise altered for use or association with another side of the body for a same or similar purpose without departing from the spirit and scope of the disclosure. For example, the instrumentation, system, and methods, and the aspects, components, features and the like thereof, described herein with respect to the right leg may be mirrored so that they likewise function with the left leg. Further, the instrumentation, system, and methods, and the aspects, components, features and the like thereof, disclosed herein are described with respect to the leg for brevity purposes, but it should be understood that the instrumentation, system, and methods may be used with other bones of the body having similar structures.

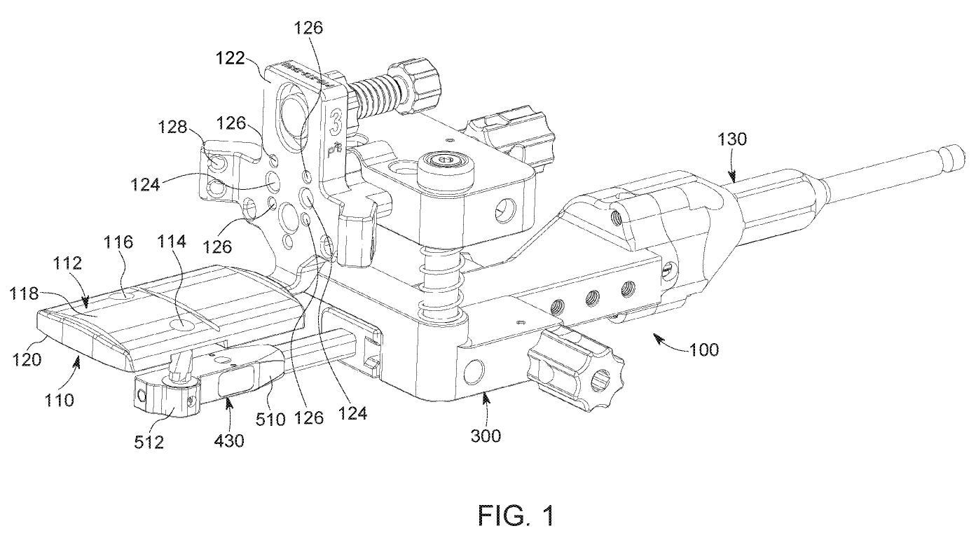

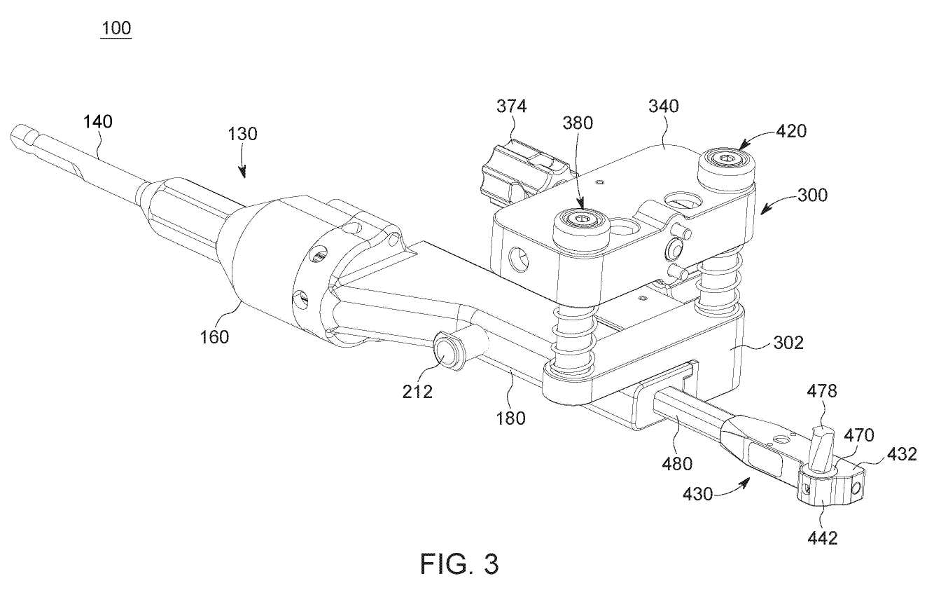

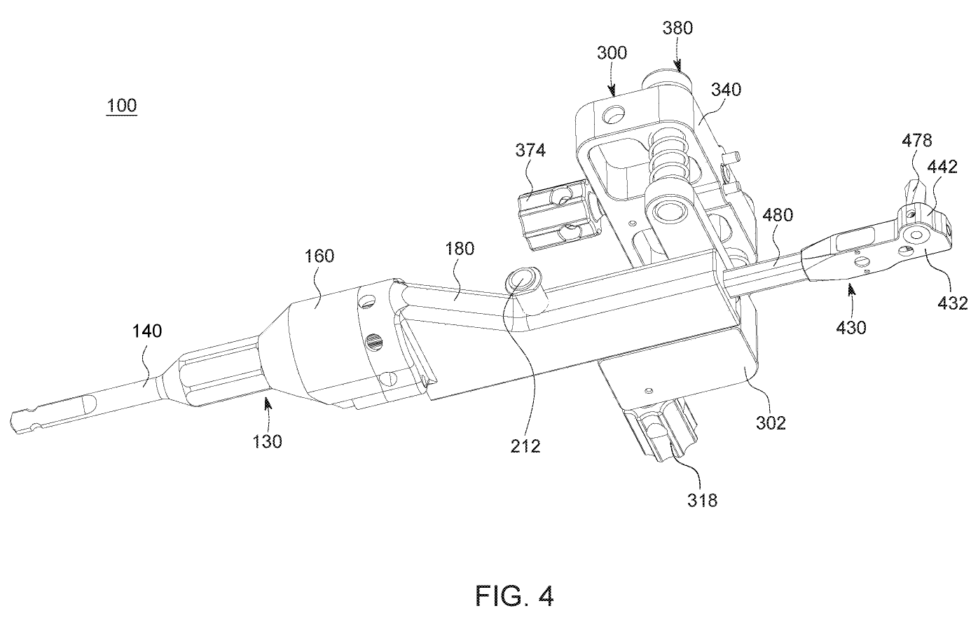

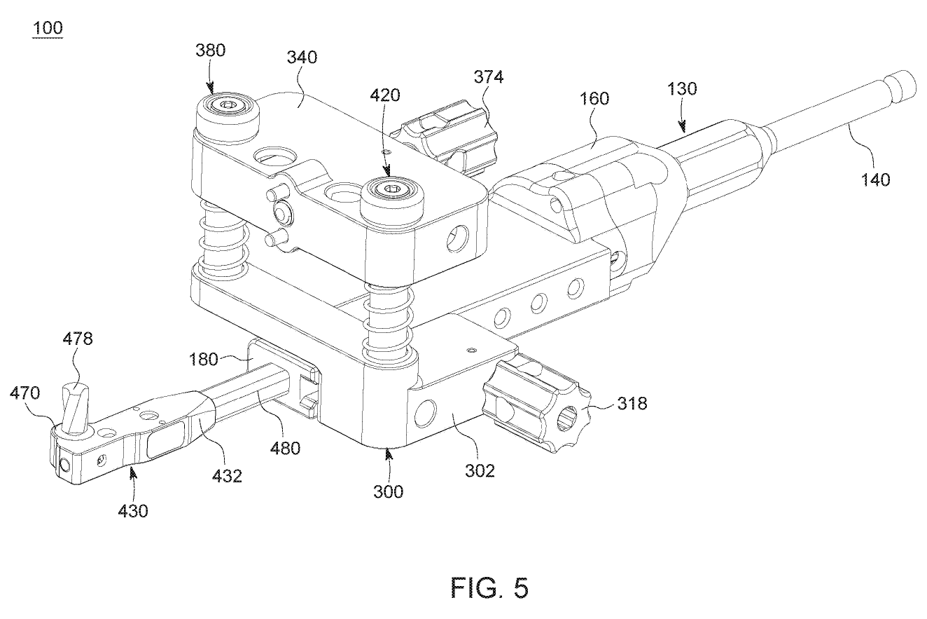

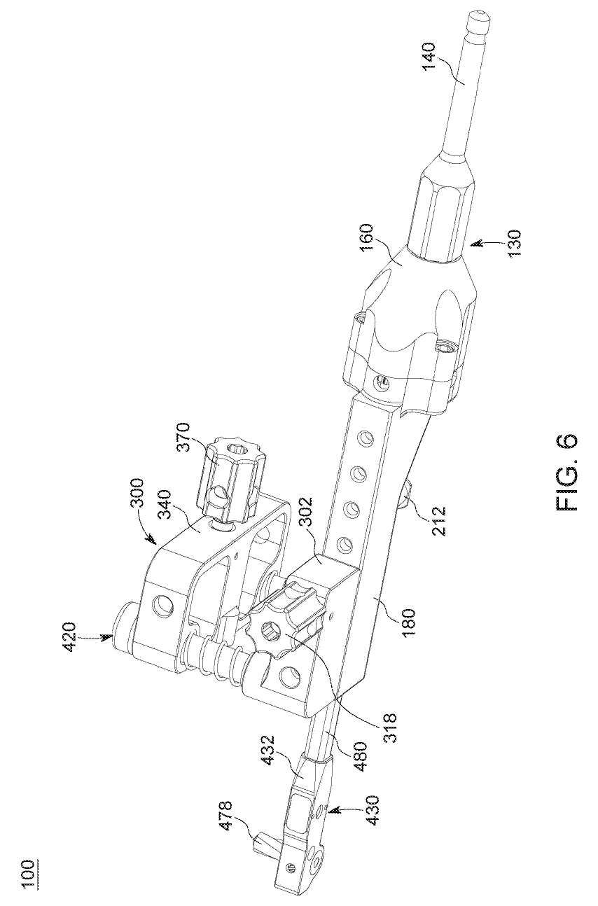

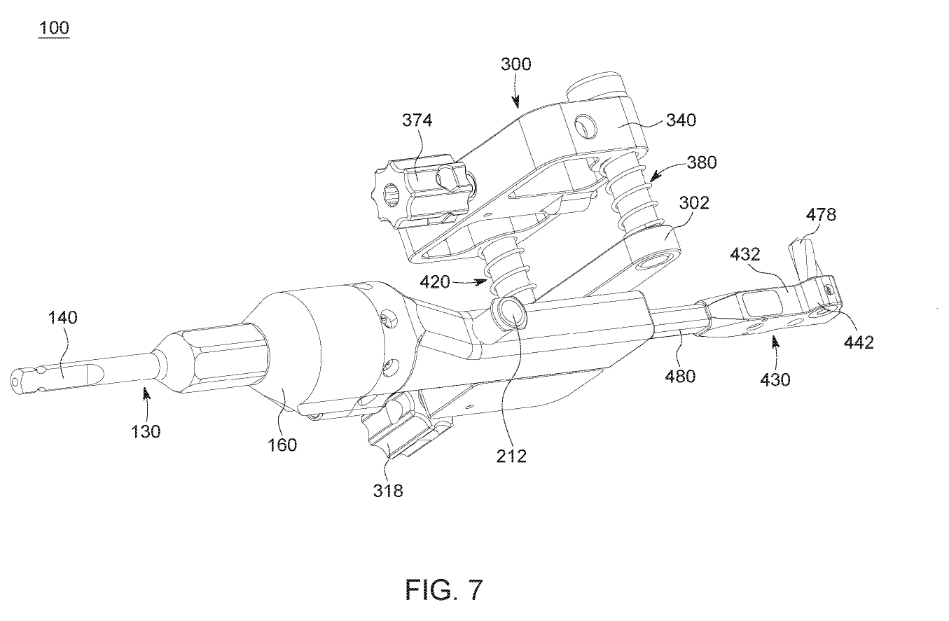

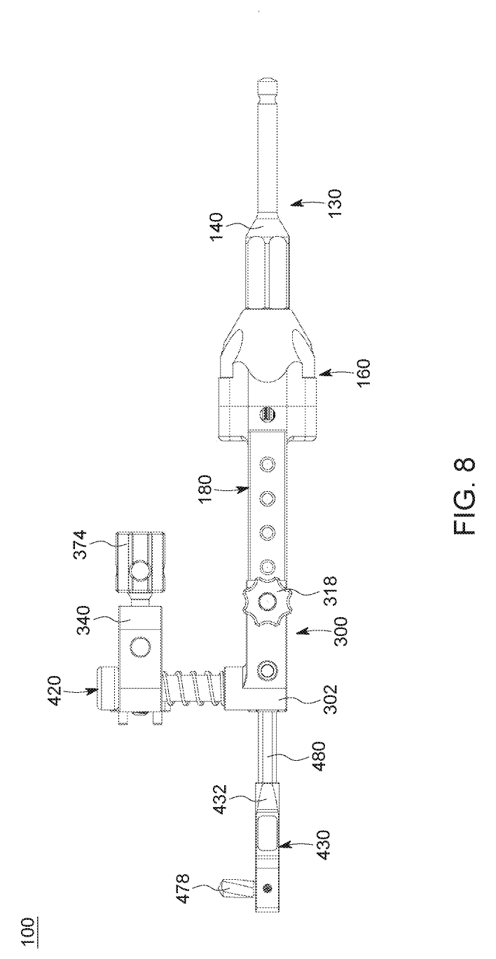

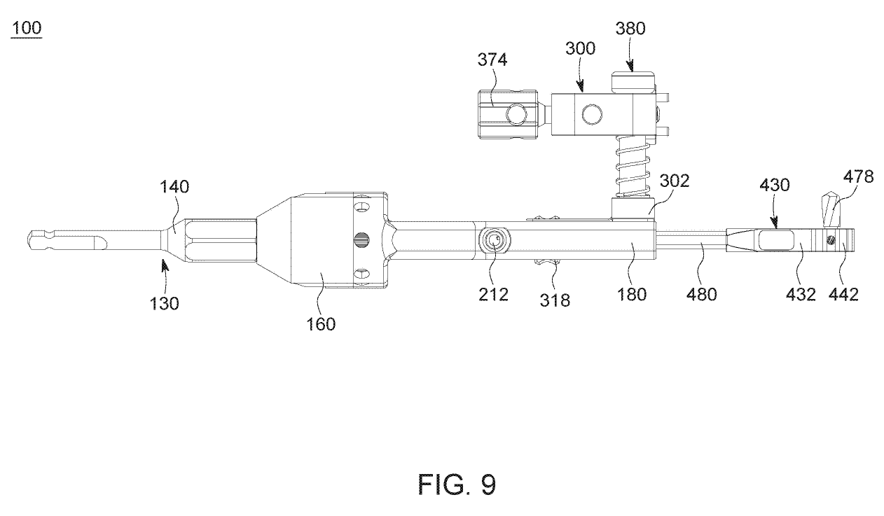

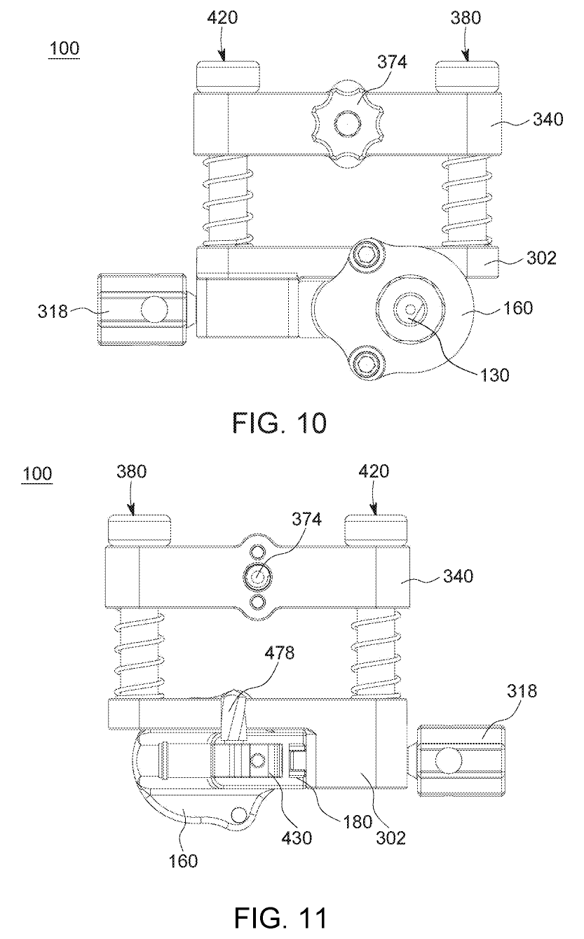

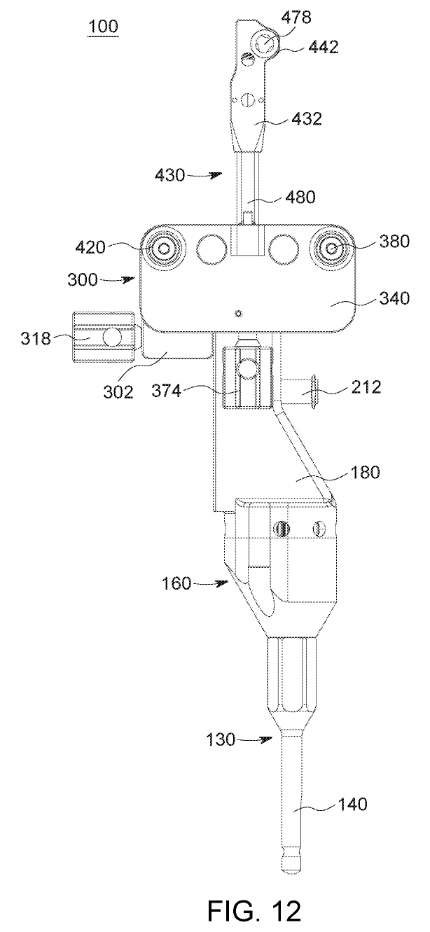

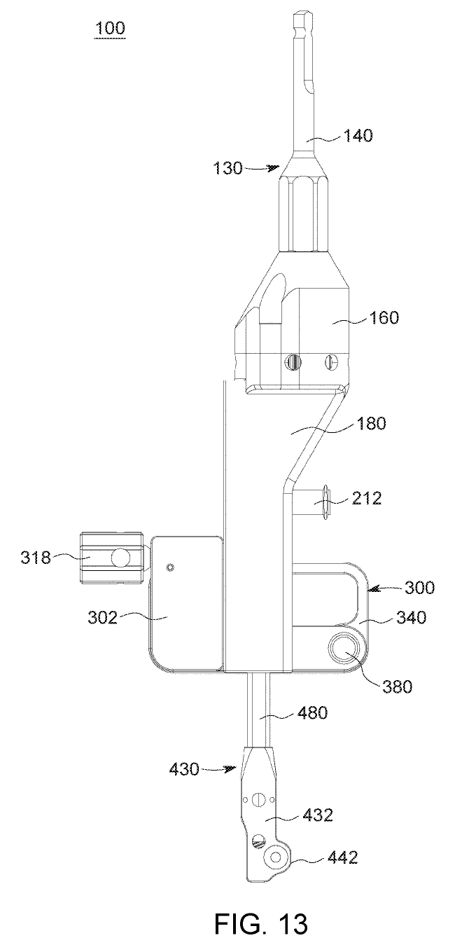

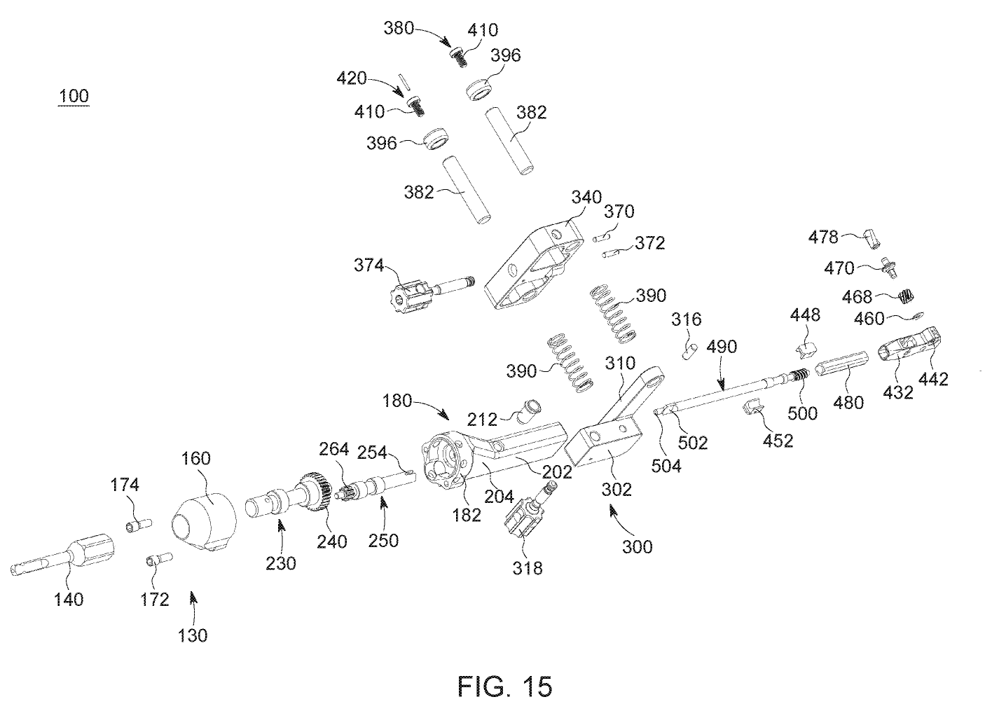

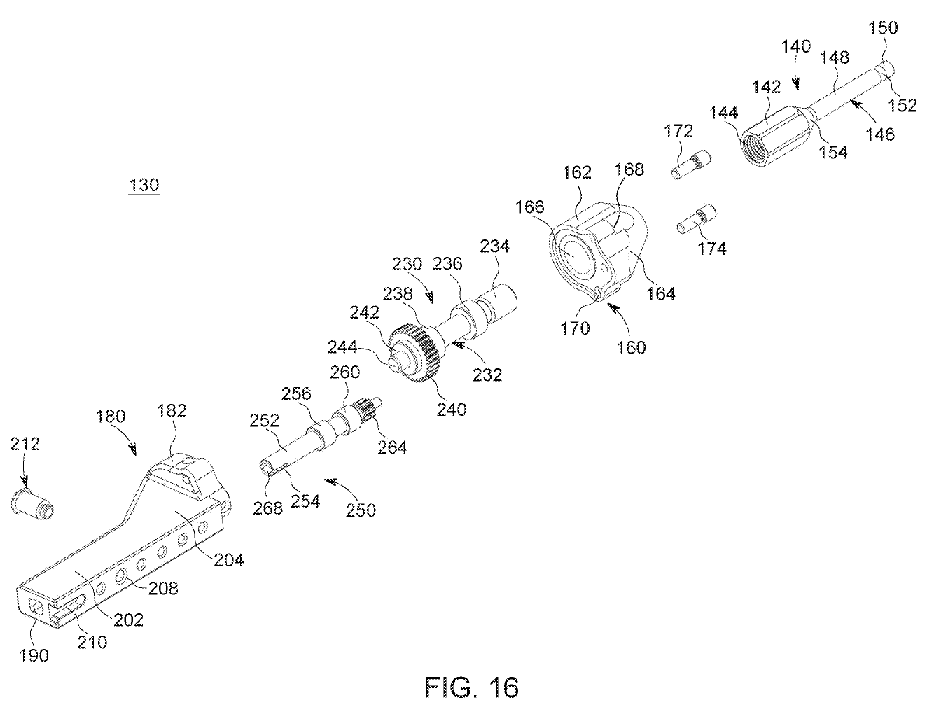

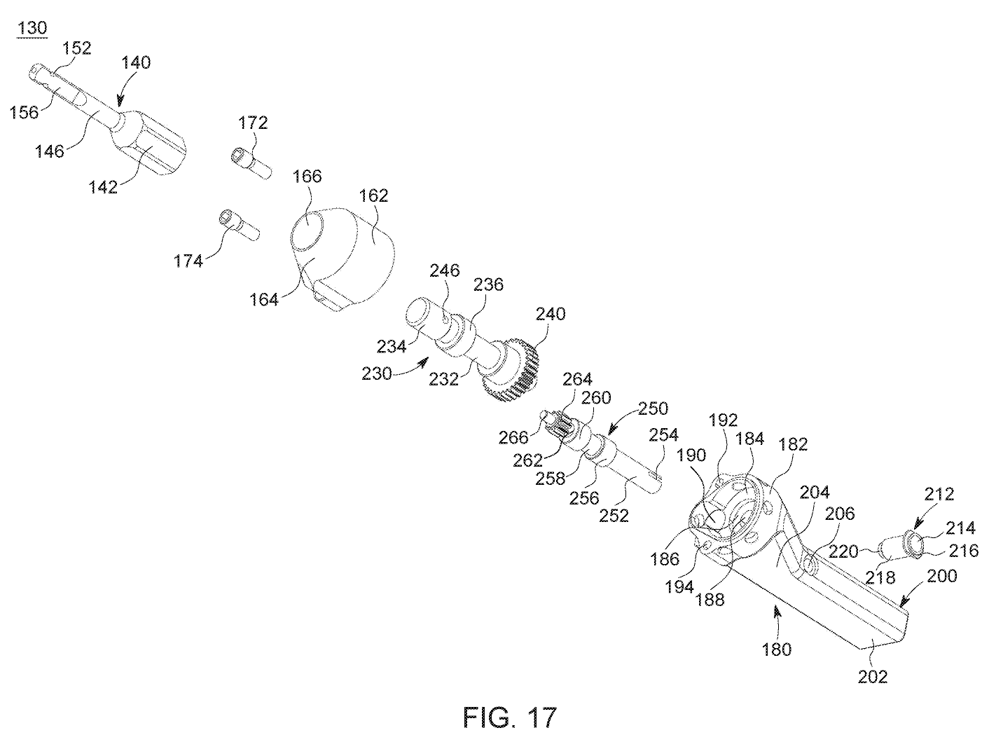

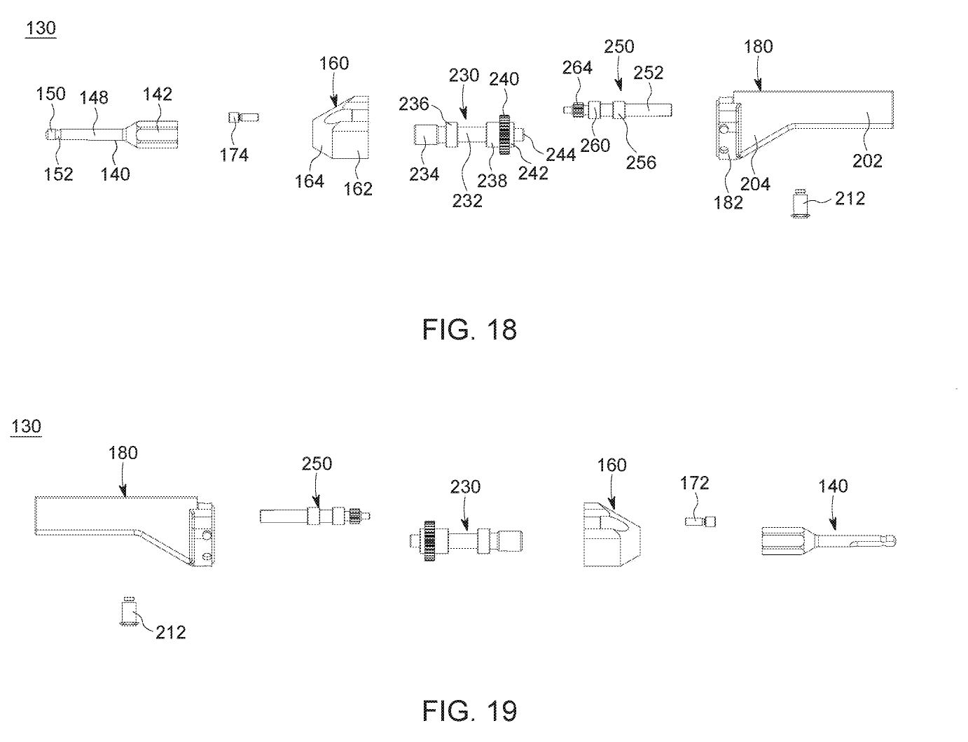

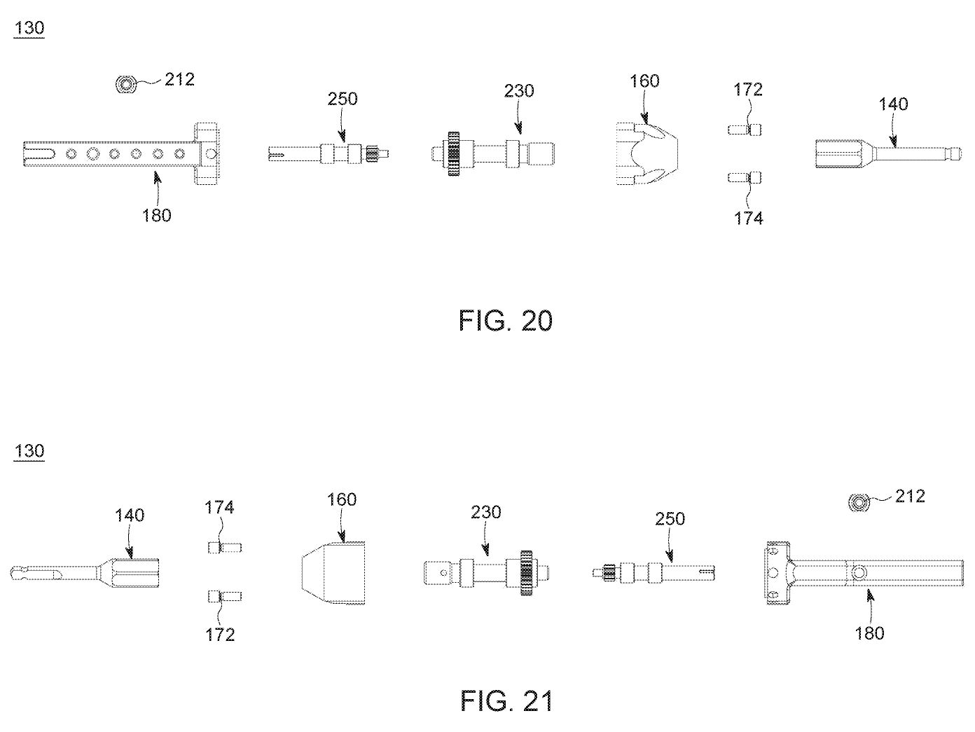

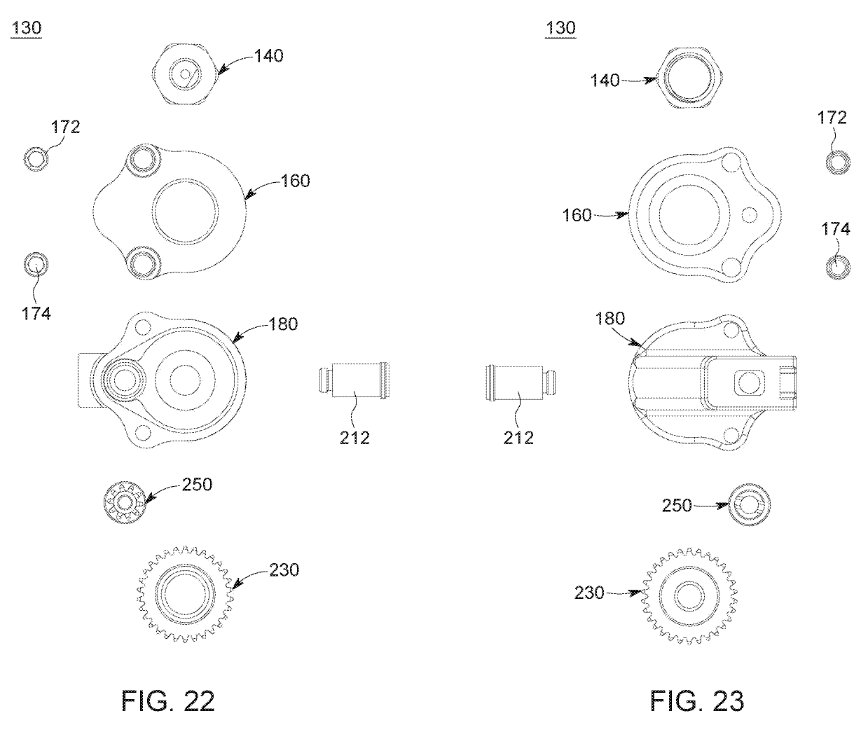

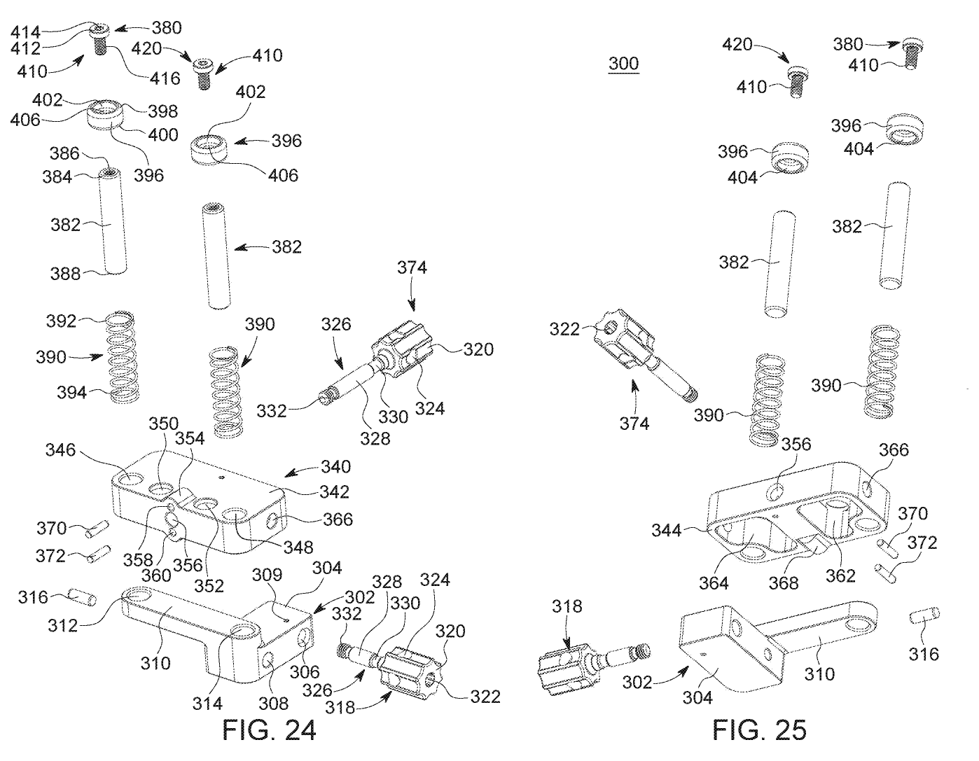

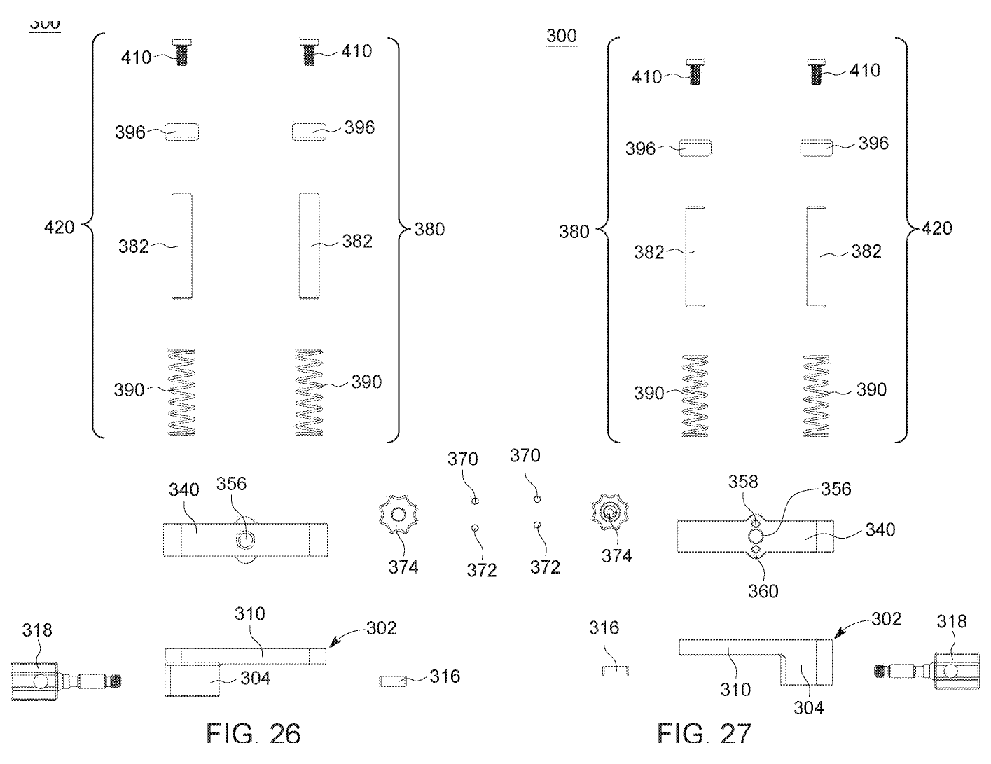

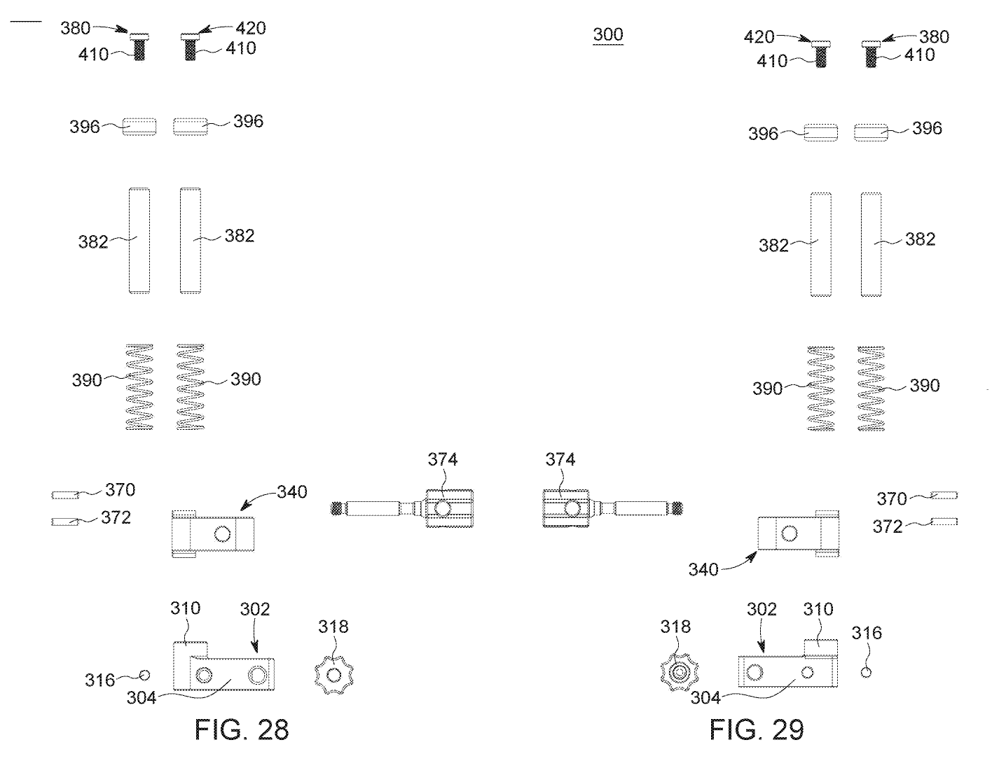

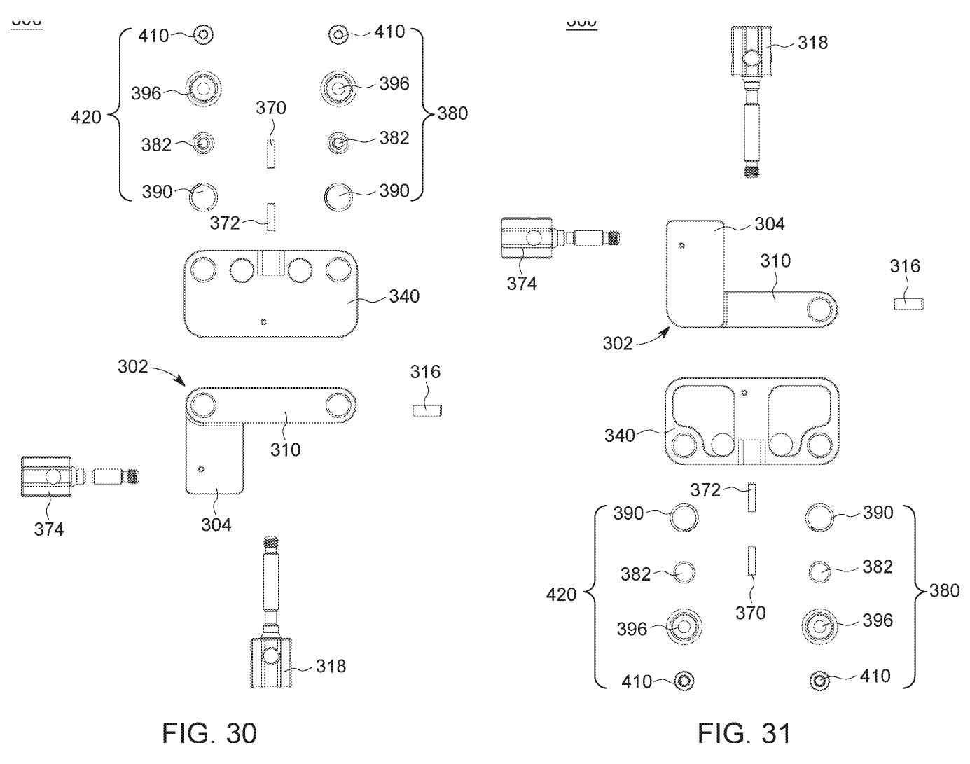

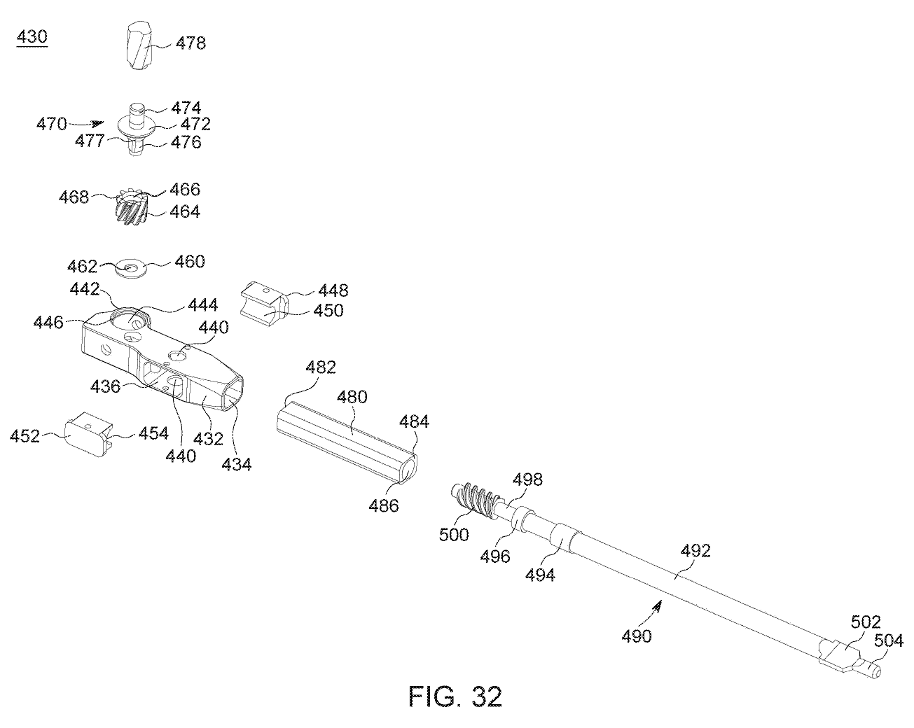

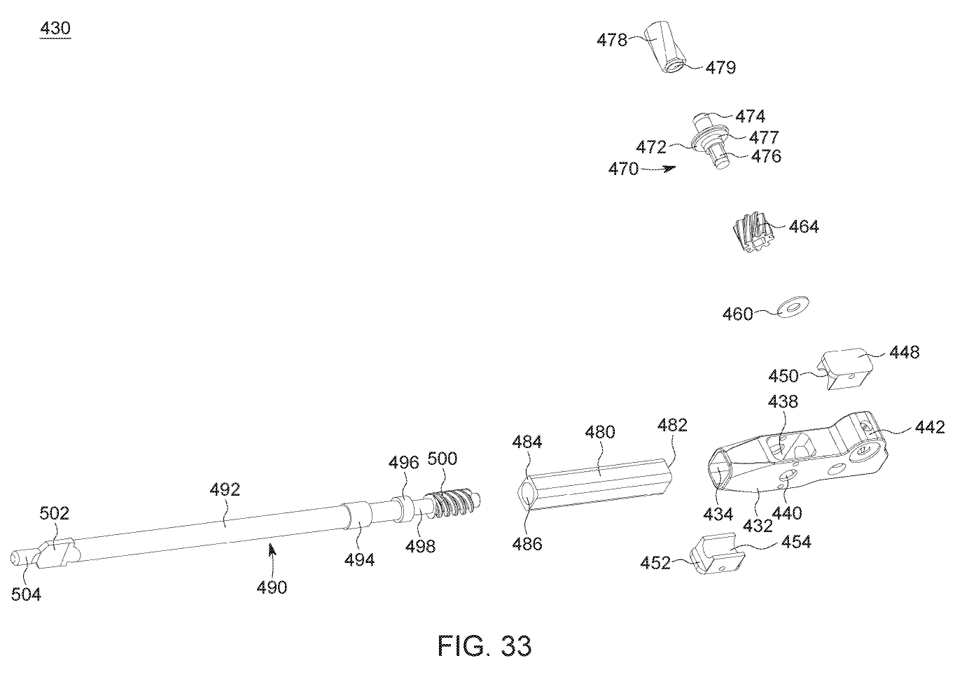

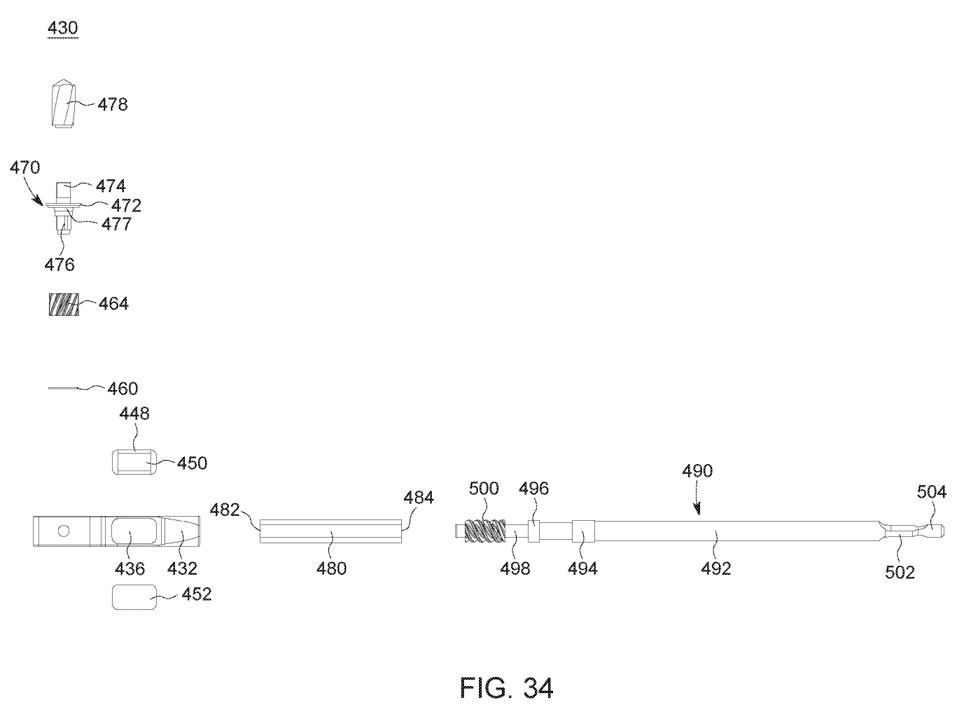

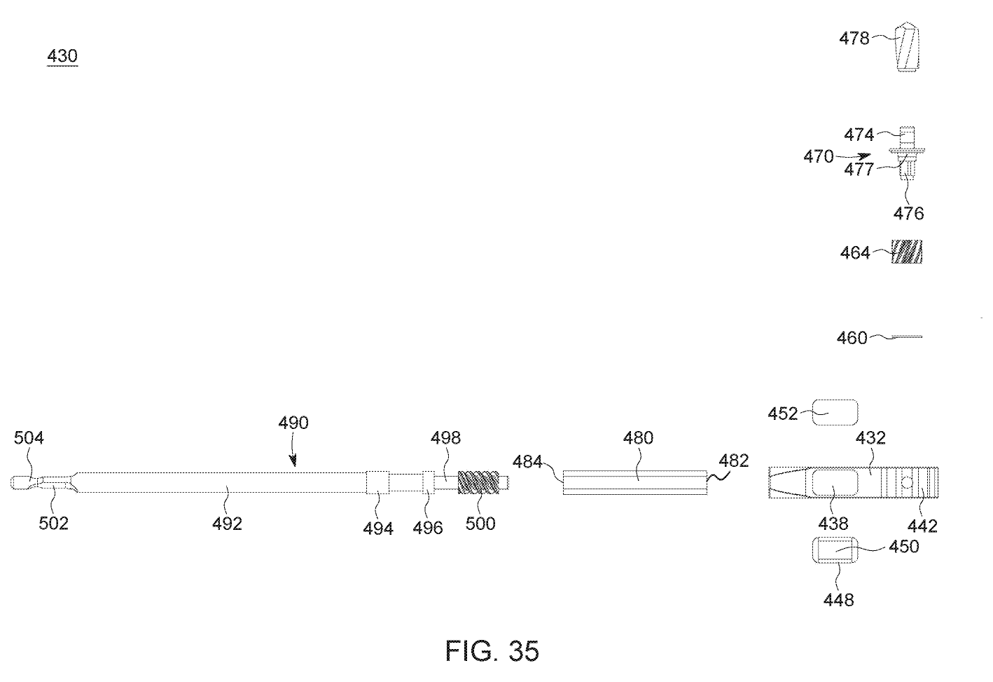

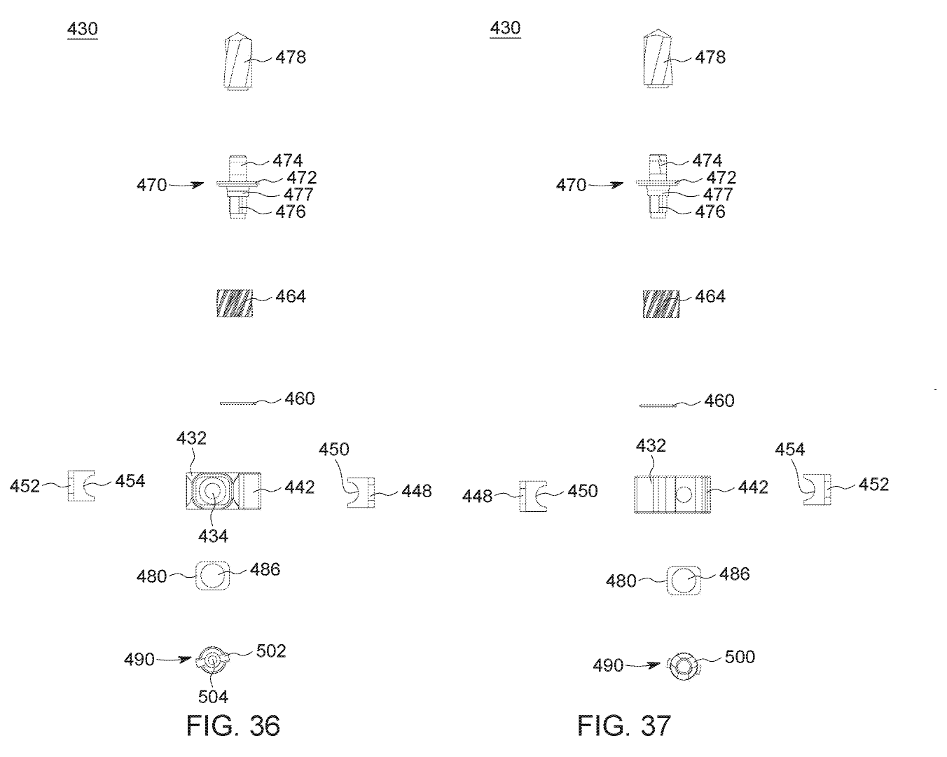

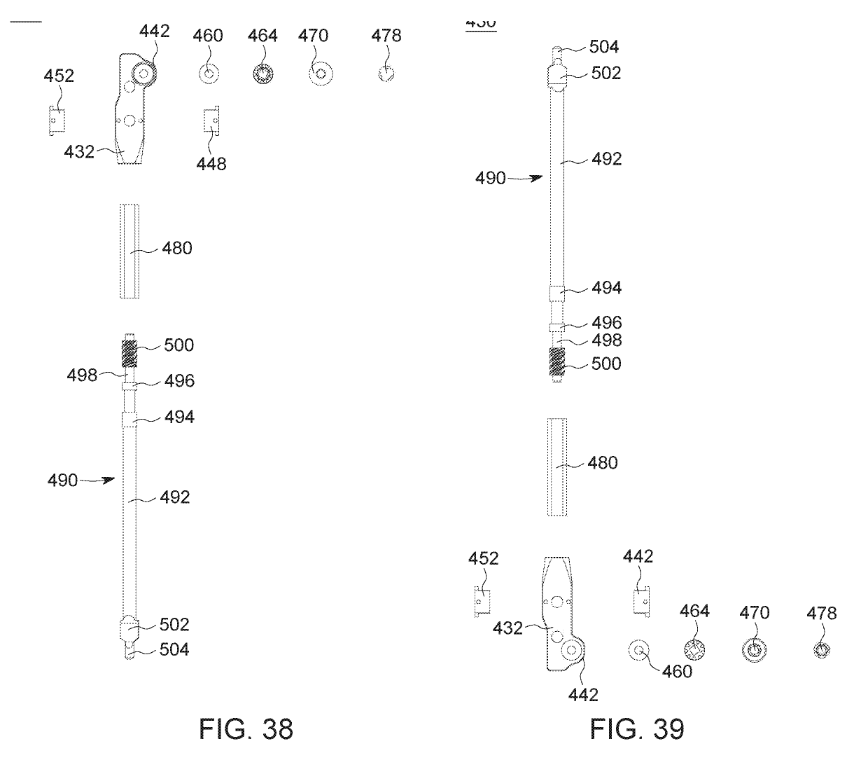

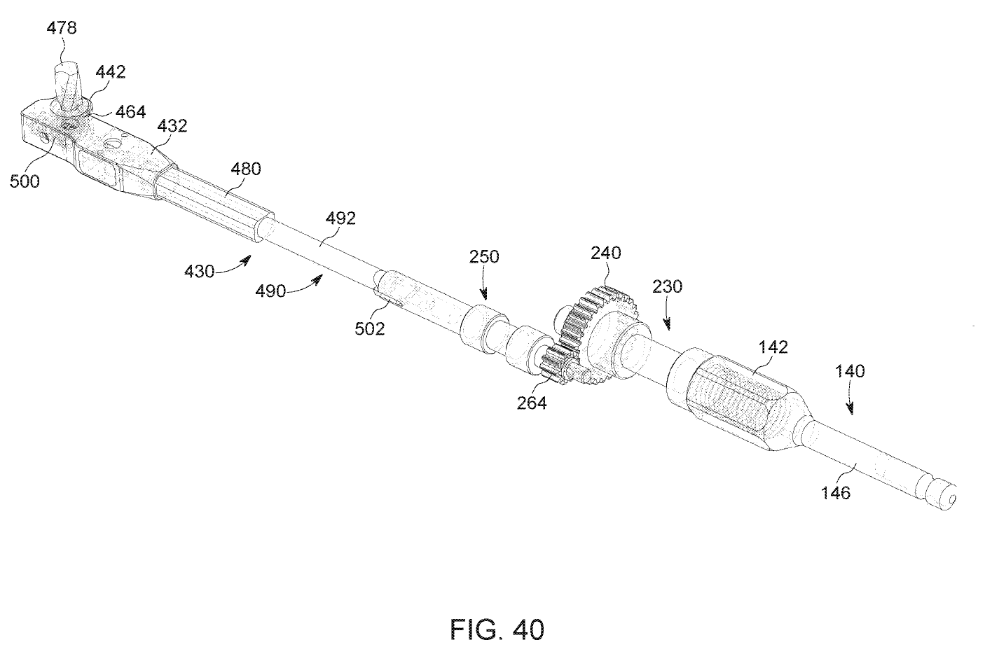

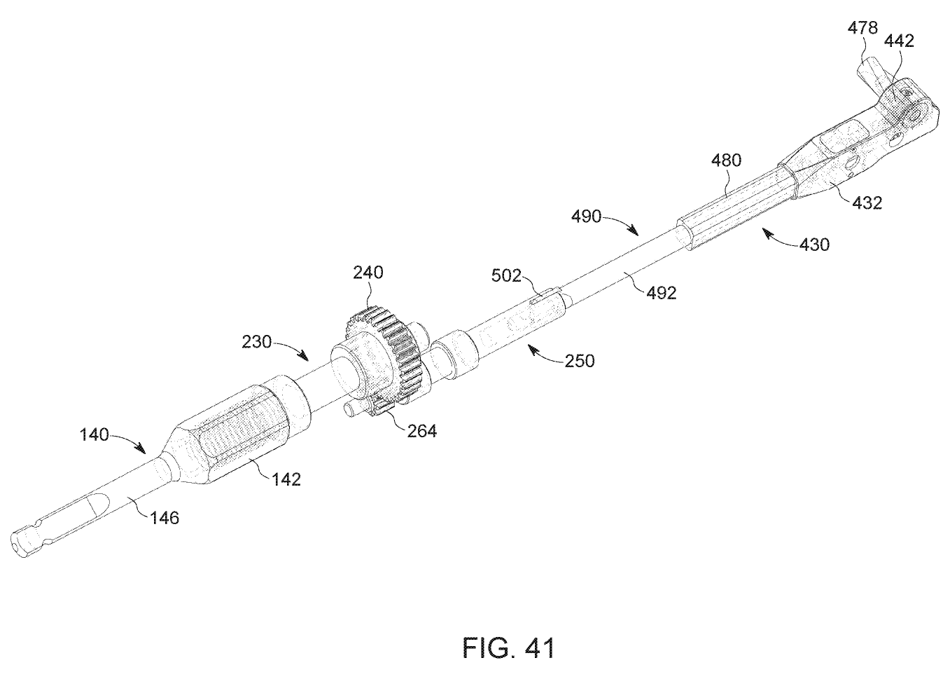

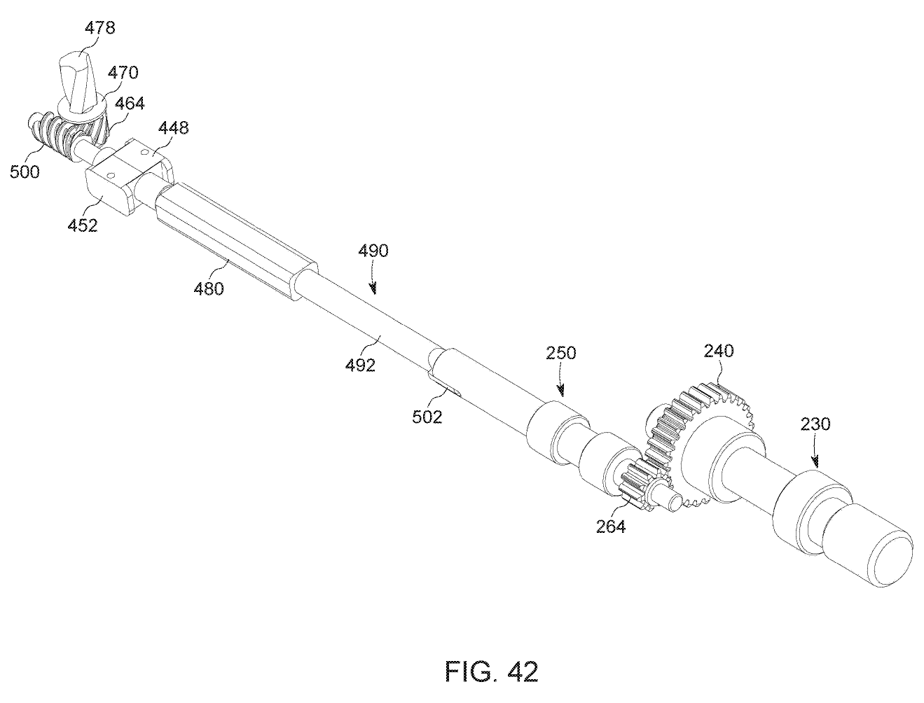

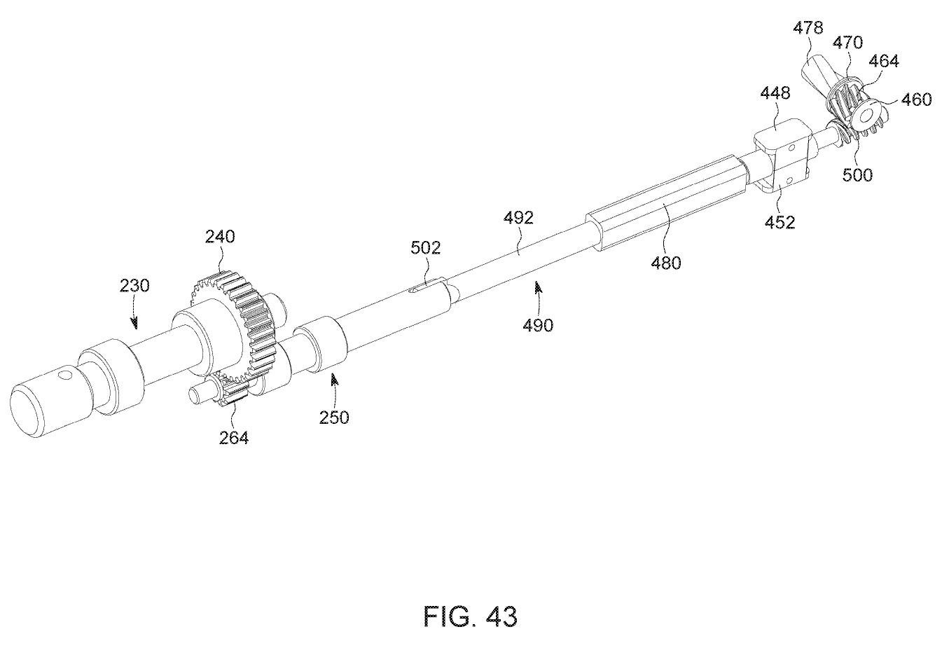

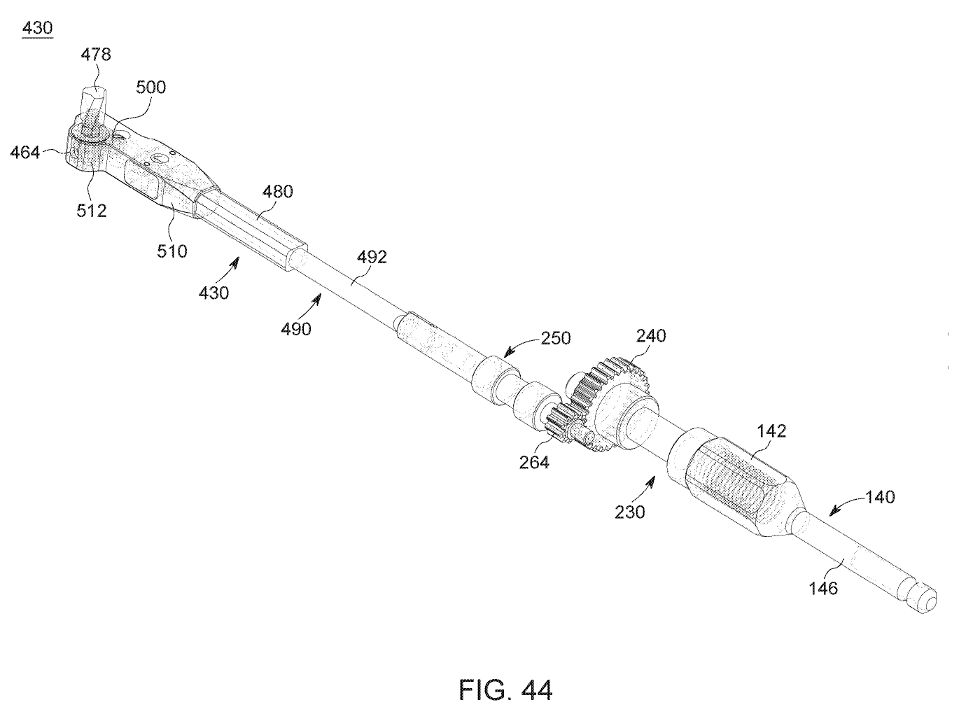

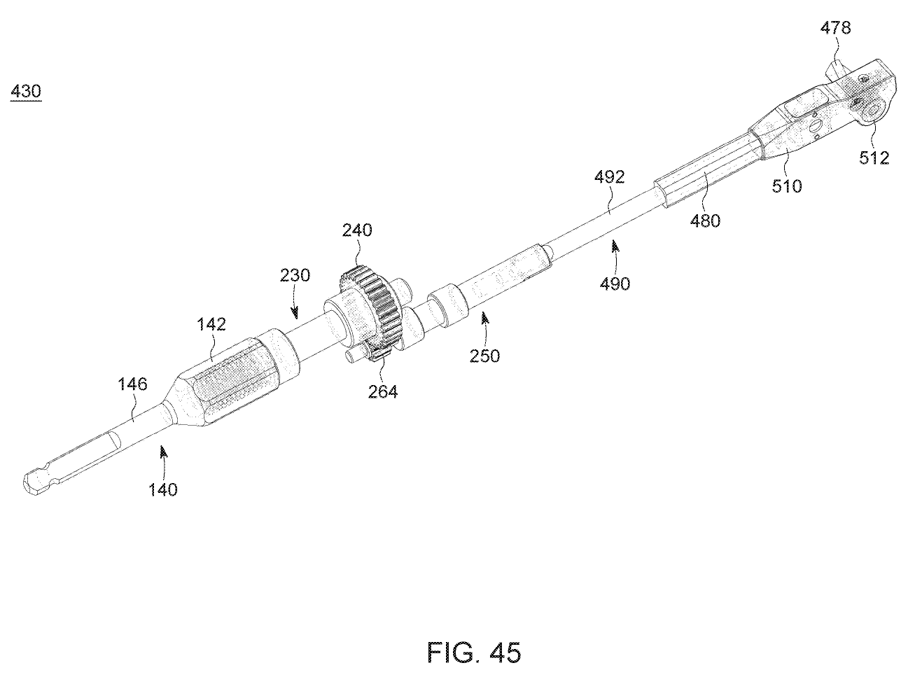

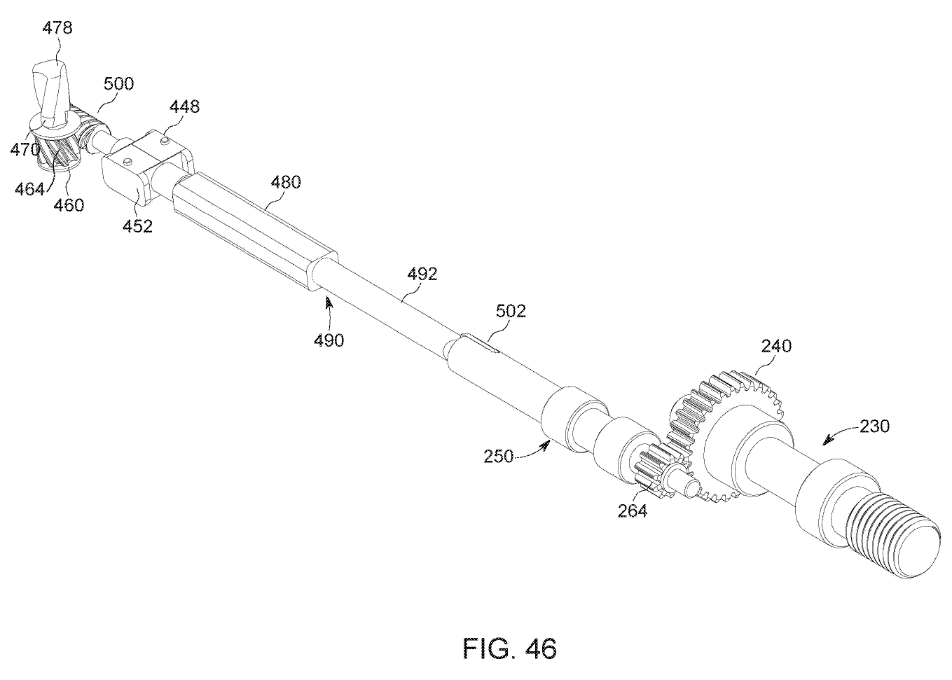

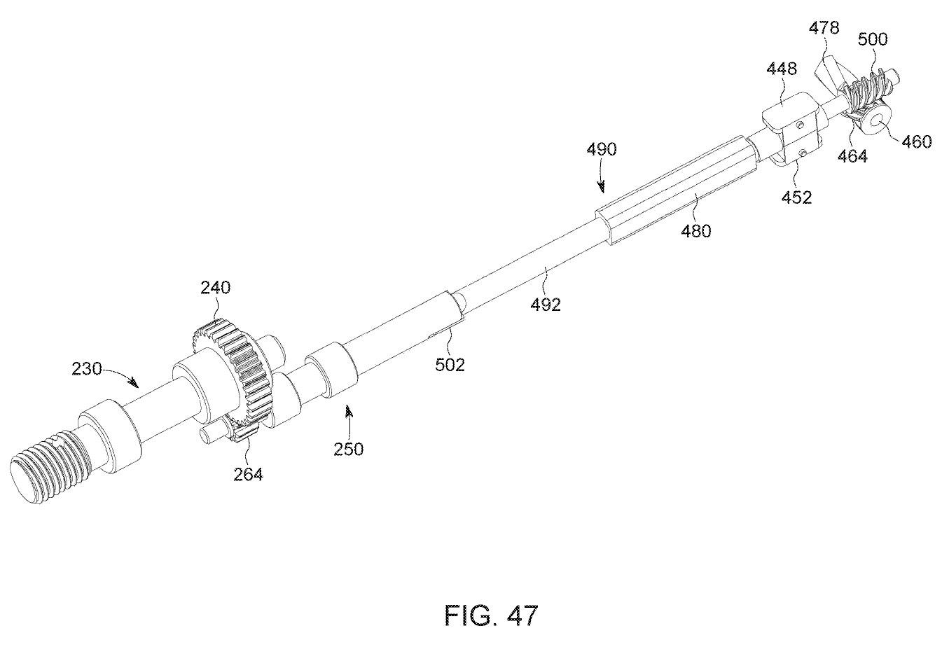

Referring to the drawings, wherein like reference numerals are used to indicate like or analogous components throughout the several views, and with particular reference to FIGS. 1-47, instrumentation, system, and methods of using the instrumentation, system, and methods for arthroplasty procedures of the ankle joint are shown.

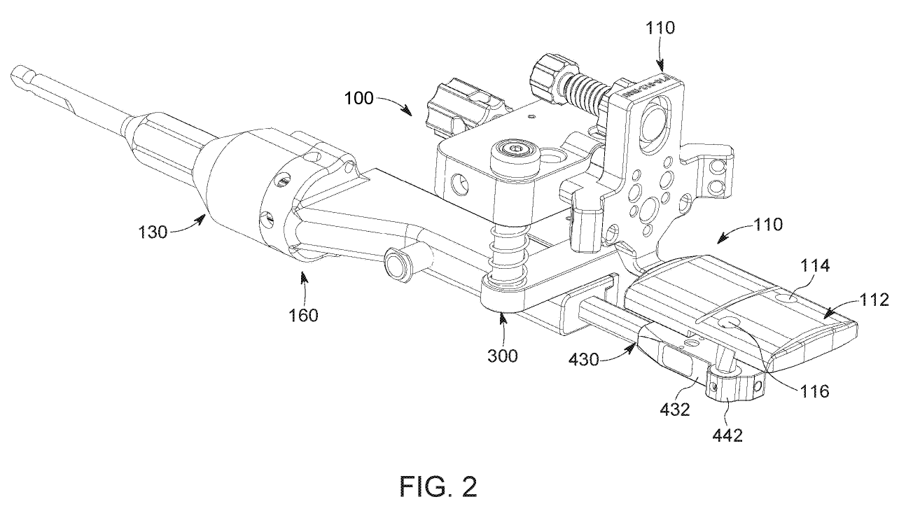

Referring now to FIGS. 1 and 2, a first instrument 100 and a second instrument 100 each coupled to a tibial cutting guide 110 are shown. The first instrument 100 with a left sided drill bit 478 is shown in FIG. 1. The second instrument 100 with a right sided drill bit 478 is shown in FIG. 2. The instruments 100 couple to the tibial cutting guide 110. The cutting guide 110 includes a tibial tray 112 and a tibial coupling element 122 that is coupled to the tibial tray 112, for example, perpendicularly or at a right angle. The tibial tray 112 includes a first drill opening 114 and a second drill opening 116. The drill openings 114, 116 may extend through the first or top surface 118 to the second or bottom surface 120. The drill openings 114, 116 may be positioned near a midpoint of the tibial tray 112. The first drill opening 114 may be spaced apart from the second drill opening 116. The first drill opening 114 may be positioned near a first side of the tibial tray 112, while the second drill opening 116 may be positioned near a second side of the tibial tray 112. The drill openings 114, 116 may be, for example, configured or sized and shaped to receive the drill bit 478 of the instruments 100. The tibial coupling element 122 may include at least one attachment opening 124 for coupling the instrument 100 to the tibial cutting guide 110. The tibial coupling element 122 further includes at least one alignment opening 126 for aligning the tibial cutting guide 110 and the instrument 100.

Referring now to FIGS. 1-47, embodiments of an instrument, drill or right angle drill 100 are shown. The instrument 100 includes a drill coupling member 140, an alignment assembly 300, and a driving member 430. The alignment assembly 300 may be positioned between the drill coupling member 140 and the driving member 430. The drill coupling member 140 couples with a drill for rotating the drill bit 478. The alignment assembly 300 allows for positioning and translating the driving member 430 relative to the tibial cutting guide 110. The driving member 430 allows for drilling of openings through the tibial cutting guide 110 for receiving a tibial implant (not shown).

With continued reference to FIGS. 1-23, the coupling portion or drill engagement portion 130 is shown. The coupling portion 130 has a first end 132 and a second end 134. The first end 132 of the coupling portion 130 couples to the alignment assembly 300. The second end 134 of the coupling portion 130 couples to a drill (not shown). The coupling portion 130 includes a drill coupling member 140, a drive housing 160 positioned adjacent to and coupled to the coupling member 140, and an angled block 180 that couples to the drive housing 160. The coupling portion 130 also includes a first drive member 230 and a second drive member 250. The drive members 230, 250 are received within the drill coupling member 140, the drive housing 160 and the angled block 180.

The drill coupling member 140, as shown in FIGS. 1-23, includes a head portion 142 and a shaft 146 extending from the head portion 142. The head portion 142 may include planar portions positioned around the circumference of the head portion 142. The head portion 142 includes an opening or threaded opening 144 extending into the head portion 142. The opening 144 may receive a portion of the first drive member 230. The shaft 146 may include a first portion 148 and a second portion 150 separated by the groove 152. The first portion 148 may be, for example, larger than the second portion 150. The shaft 146 may also include a tapered region 154 where the shaft 146 couples to the head portion 142. The shaft 146 may further include an alignment portion 156 inset into a portion of the shaft 146. The alignment portion 156 may assist with insertion of the shaft 146 into the drill.

The drive housing 160 may include a body 162 and a tapered portion 164 extending and tapering away from the body 162, as shown in FIGS. 1-23. The body 162 may have planar portions and curved or rounded portions around the circumference of the body 162. The drive housing 160 may also include a through hole 166 extending from the first end to the second end of the drive housing 160 through the body 162 and the tapered portion 164. The drive housing 160 may also include a first screw hole 168 and a second screw hole 170 extending through a portion of the body 162 to the first end of the drive housing 160. A portion of the screw holes 168, 170 may be, for example, recessed into a portion of the tapered portion 164. The first and second screw holes 168, 170 may be, for example, configured or sized and shaped to receive a first and second fastener 172, 174, respectively. The fasteners 172, 174 may be, for example, screws, bolts, or the like. The fasteners 172, 174 may extend through the screw holes 168, 170 to secure the drive housing 160 and the angled block 180.

FIGS. 1-23 also show the block or angled block 180 with a housing 182 and a base 200 coupled to and extending from the housing 182. The housing 182 may have, for example, a first recessed region 184 extending into the block 180 from a second end into the housing 182. The housing 182 may also include a second recessed region 186 inset into the housing 182 from the first recessed region 184 toward the first end of the block 180. The second recessed region 186 may have, for example, a circumference smaller than the circumference of the first recessed region 184. The housing 182 may further include a third recessed region 188 inset into the housing 182 from the second recessed region 186 toward the first end of the block 180. The third recessed region 188 may have, for example, a circumference smaller than the circumference of the second recessed region 186. Thus, the circumference of the first recessed region 184 is larger than the circumference of the third recessed region 188. The block 180 also includes a through hole 190 extending through the housing 182 and the base 200 from the first end of the block 180 to the second end. The through hole 190 may be positioned near a first side of the block 180. The through hole 190 may, for example, overlap with the first recessed region 184. The housing 182 further includes a first screw hole 192 and a second screw hole 194. The screw holes 192, 194 may be positioned on opposite sides of the through hole 190. The screw holes 192, 194 may be, for example, positioned to engage the fasteners 172, 174 after the fasteners 172, 174 pass through the drive housing 160.

The base 200 may include a first portion 202 and a second or tapered portion 204. The tapered portion 204 extends between the housing 182 and the first portion 202. The housing 182 may be, for example, wider than the width of the first portion 202. The first portion 202 may include an opening or luer port opening 206 extending from a first side of the base 200 to the through hole 190. The base 200 may further include a plurality of through holes 208 extending through the base 200 from a second side to the through hole 190. The base 200 may also include a slot 210 extending into the second side of the first portion 202 from a first end toward the second end. The slot 210 may extend into the base 200 parallel to the through hole 190. The opening 206 may receive, for example, a port or luer port 212 to connect tubing or a syringe to the base 200 and access the through hole 190. The port 212 may include a through hole 214 extending through the port 212 from the first end to the second end. The port 212 may also include a head 216 and a shaft 218 extending away from the head 216. In addition, the port 212 may include a connector 220 to couple the port 212 to the base 200.

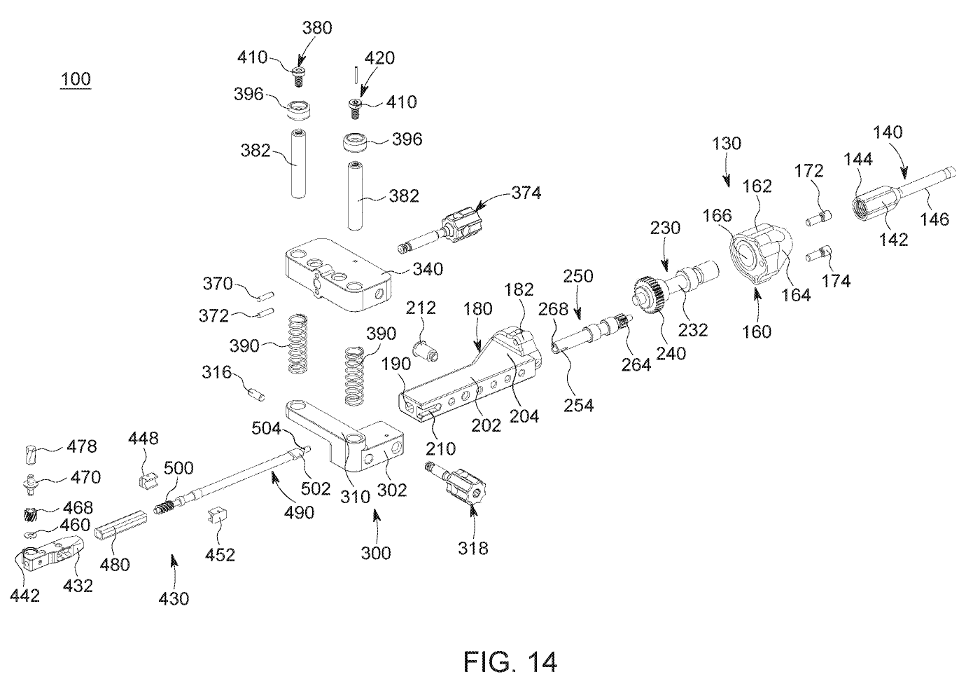

As shown in FIGS. 14-23, the first drive member 230 includes a shaft 232 with a first gear 240 at a first end. The shaft 232 includes a first protrusion 234 extending from the shaft 232 at the second end. The shaft 232 includes a second protrusion 236 extending from the shaft 232 between the first protrusion 234 and the first gear 240. The second protrusion 236 may be, for example, spaced from the first protrusion 234. In addition, the shaft 232 includes a third protrusion 238 extending from the shaft 232 near or adjacent to the first gear 240. The third protrusion 238 is positioned between the second protrusion 236 and the first gear 240. The shaft 232 may include a fourth protrusion 242 positioned near the first end of the shaft 232. The fourth protrusion 242 may be positioned near or adjacent to the first gear 240. The fourth protrusion 242 is positioned between the first gear 240 and the first end of the shaft 232. The first gear 240 is positioned near the first end of the shaft 232. The first gear 240 may have, for example, a circumference larger than the circumferences of the protrusions 234, 236, 238, 242. The first gear 240 may be positioned between the third protrusion 238 and the fourth protrusion 242. The drive member 230 may also have a head portion 244 positioned at the first end of the drive member 230. The head portion 244 may have a diameter smaller than the diameter of the shaft 232. Further, the drive member 230 may include a locking hole 246 extending into the first protrusion 234 for receiving a pin or securement member to secure the first drive member 230 to the coupling member 140.

With continued reference to FIGS. 14-23, the second drive member 250 is shown. The second drive member 250 includes shaft portions 252, 258, 262, protrusions 256, 260, and a second gear 264. A first protrusion 256 is positioned between a first shaft portion 252 and a second shaft portion 258. A second protrusion 260 is positioned between the second shaft portion 258 and a third shaft portion 262. The first protrusion 256 is spaced apart from the second protrusion 260. The second gear 264 may be positioned between the third shaft portion 262 and a head portion 266. The second gear 264 may have, for example, a diameter that is the same size as or smaller than the protrusions 256, 260. The second gear 264 is configured or sized and shaped to engage the first gear 240 within the coupled drive housing 160 and block 180. The head 266 may have a diameter smaller than the diameters of the shaft portions 252, 258, 262, the protrusions 256, 260, and the second gear 264. The second drive member 250 may also include a channel 254 extending into the first shaft portion 252 from the first end. The second drive member 250 may further include an opening 268 extending into the first shaft portion 252 from the first end toward the second end of the second drive member 250. The channel 254 may, for example, bisect the opening 268.

The alignment assembly 300, as shown in FIGS. 1-15 and 24-31, includes a first or bottom alignment bar 302, a second or top alignment bar 340, and a first coupling system 380 and a second coupling system 420 for movably coupling the first alignment bar 302 to the second alignment bar 340. The first alignment bar 302 includes a base 304 and an extension member 310 extending away from and perpendicular to a first end of the bar 304. The extension member 310 is coupled to a top surface of the base 304. The base 302 includes a first through hole 306 and a second through hole 308, each extending through the base 302 from a first side to a second side. The first through hole 306 may be, for example, positioned near a second end of the base 302. The second through hole 308 may be, for example, positioned near a first end of the base 302. The base 302 may also include a third through hole 309 extending through the base 302 from a top surface to a bottom surface. The third through hole 309 may, for example, engage at least a portion of the first through hole 306. The second through hole 308 receives at least a portion of an alignment peg 316. The extension member 310 is an elongated member with rounded ends. The extension member 310 includes a through hole 312 at a first or right side and an opening 314 at a second or left side. The through hole 312 extends through the extension member 310 from a first or top surface to a second or bottom surface. The opening 314 extends from the top surface through the extension member 310 to the base 304.

With continued reference to FIGS. 1-15 and 24-31, the alignment assembly 300 also includes a first securement knob 318. The first securement knob 318 includes a head 320 and a shaft 326 extending away from the head 320. The head 320 includes a drive opening 322 extending into the head 320 from the first end. The head 320 also includes through holes 324 near a second end of the knob 318 and extending through the knob 318 distal to the opening 322. The shaft 326 includes a first or central portion 328 extending from a second end of the knob 318. The shaft 326 also includes a recess 330 inset into the shaft 326 near a first end of the shaft 326 and the second end of the knob 318. The shaft 326 further includes a threaded end 332 at a second end of the shaft 326. The central portion 328 of the shaft 326 is positioned between the recess 330 and the threaded end 332. The third through hole 309 may receive a pin (not shown) for engaging and securing the first securement knob or knob 318 within the base 302, while allowing for rotation of the knob 318. The pin (not shown) may engage, for example, the recess 330 of the first securement knob 318.

As shown in FIGS. 1-15 and 24-31, the second alignment bar 340 includes a top surface 342 and a bottom surface 344. The second alignment bar 340 also includes a first through hole 346 and a second through hole 348. The first through hole 346 is positioned at a first corner at the first side and first end of the second alignment bar 340. The second through hole 348 is positioned at a second corner at the second side and first end of the second alignment bar 340. The through holes 346, 348 extend through the second alignment bar 340 from the top surface 342 to the bottom surface 344. The second alignment bar 340 may also include a first opening 350, a second opening 352, a first protrusion 354, and a second protrusion 368. The protrusion 354 may be, for example, positioned at or near a midpoint of the first end of the second alignment bar 340. The first opening 350 is positioned between the first through hole 346 and the protrusion 354. The second opening 352 is positioned between the second through hole 348 and the protrusion 354. The second alignment bar 340 also includes a securement opening 356 extending from the first end to the second end of the second alignment bar 340. The securement opening 356 is configured to receive a second securement knob 374. The second securement knob 374 may be, for example, the same as or similar to the first securement knob 318 and which will not be described in detail here for brevity’s sake. The second alignment bar 340 also includes a first alignment pin opening 358 and a second alignment pin opening 360. The alignment pin openings 358, 360 extend into a portion of the second alignment bar 340 from a first end. The first and second pin openings 358, 360 are configured or sized and shaped to receive first and second alignment pins 370, 372, respectively. The first alignment pin opening 358 is positioned superior to or above the securement opening 356. The first pin opening 358 may extend into a portion of the first protrusion 354. The second alignment pin opening 360 is positioned inferior to or below the securement opening 356. The second pin opening 360 may extend into a portion of the second protrusion 368.

The second alignment bar 340 also includes a first recess 362 extending into the alignment bar 340 from the bottom surface 344 toward the top surface 342. The second alignment bar 340 also includes a second recess 364 extending into the alignment bar 340 from the bottom surface 344. The first recess 362 is positioned on a first side of the alignment bar 340 and the second recess 364 is positioned on a second side of the alignment bar 340. The second alignment bar 340 may also include a second protrusion 368 extending from a bottom surface 344 of the second alignment bar 340. The second protrusion 368 may be positioned, for example, at a midpoint of the first end of the second alignment bar 340. The second protrusion 368 may be, for example, positioned opposite and/or as a mirror image of the first protrusion 354. The second alignment bar 340 also includes at least one hole 366 extending through the alignment bar 340 on a first side and a second side.

The first alignment bar 302 is coupled to the second alignment bar 340 by the first and second coupling systems 380, 420. As shown in FIGS. 1-15 and 24-31, the first coupling system 380 includes a rod 382, a spring 390, a retaining member 396, and a screw 410. The rod 382 includes a first end 384 and a second end 388. The first end 384 includes an opening or threaded opening 386 for receiving a portion of the screw 410. The second end 388 of the rod 382 is received within the through hole 312 of the first alignment bar 302. The spring 390 is positioned or received circumferentially around the rod 382. The spring 390 includes a first end 392 and a second end 394. The first end 392 of the spring 390 engages the second alignment bar 340 and the second end 394 of the spring 390 engages the first alignment bar 302. The retaining member 396 includes a first end or top 398 and a second end or bottom 400. The retaining member 396 also includes a first recess 402 extending into the first end 398 of the retaining member 396 and a second recess 404 extending into the second end 400 of the retaining member 396. The recesses 402, 404 are, for example, configured or sized and shaped to receive the first end 384 of the rod 382. The retaining member 396 also includes a through hole 406 extending through the retaining member 396 from a first end to a second end. The through hole 406 extends, for example, through a central axis or center point of the retaining member 396.

The screw 410 includes a head 414 and a shaft 416 extending from the head 414. The head 414 includes threads along at least a portion of the shaft 416. The shaft 416 may be received within and coupled to the threaded opening 386 of the rod 382. The head 414 may also include a drive opening 418 extending into the first end of the head. The drive opening 418 may be used for rotating the screw 410. The screw 410 is inserted through the through hole 406 of the retaining member 396 and into the threaded opening 386 of the rod 382 to secure the first coupling system 380 between the alignment bars 302, 340. The second coupling system 420 may be, for example, the same or similar to the first coupling system 380 and which will not be described again here for brevity’s sake. Once the coupling systems 380, 420 are inserted between the alignment bars 302, 340, the alignment bars 302, 340 may, for example, move relative to each other along the rods 382.

With continued reference to FIGS. 2-15 and 32-43, an instrument 100 with a right sided drive member 430 is shown. The right sided drive member 430 may include a right sided housing 432, a sleeve 480, and a drive shaft 490. The housing 432 may include a through hole 434 extending into the housing 432 from the second end toward the first end. The housing 432 may also include a first cavity 436 extending from a first side of the housing 432 into the through hole 434. The housing 432 may further include a second cavity 438 extending from a second side of the housing 432 into the through hole 434. The housing 432 may also include holes 440 extending through the housing 432 from the top surface to the bottom surface. The holes 440 may extend from the exterior of the housing into the cavities 436, 438. In addition, the housing 432 may have a protrusion 442 extending from a second side of the housing 432 near a first end. An opening 444 extends into the housing 432 from a top surface toward the second surface. At least a portion of the opening 444 extends into the protrusion. The opening 444 may also include a lip 446 surrounding the opening 444. The drive member 430 may also include a first sidewall 448 for insertion into the second cavity 438 and a second sidewall 452 for insertion into the first cavity 436. The first sidewall 448 may include a first recessed region 450 on the interior surface of the first sidewall 448 to surround the through hole 434 on a first side. The second sidewall 452 may include a second recessed region 454 on the interior surface of the second sidewall 452 to surround the through hole 434 on a second side. The sidewalls 448, 452 may be secured to the housing 432 with pins (not shown) inserted through holes in the housing 432 and sidewalls 448, 452.

The drive member 430 may further include a washer 460 received within the opening 444 and positioned on top of the lip 446. The washer 460 may include a through hole 462 extending through the washer 460 from the top surface to the bottom surface. The drive member 430 may also include a first worm gear 464 that may be positioned to rotate on top of the washer 460. The first worm gear 464 may include a recessed region 466 extending into the worm gear 464 from a top surface toward a bottom surface. The first worm gear 464 may also include a through hole 468 extending through the worm gear 464 from the top surface to the bottom surface. The through hole 468 may have, for example, a rectangular or other polygonal shape. The drive member 430 further includes a rotating member 470 with a base 472, a first shaft 474 extending away from a first or top surface of the base 472, and a second shaft 476 extending away from a second or bottom surface of the base 472. The first shaft 474 may have, for example, a round or circular shape. The second shaft 476 may have, for example, a square or polygonal shape to correspond to the shape of the through hole 468 of the first worm gear 464. The rotating member 470 may also include a protrusion 477 extending from the second shaft 476 adjacent to the base 472. The protrusion 477 is received within and engages the recessed region 466 of the first worm gear 464. Further, the drive member 430 includes a drill bit 478 with an opening 479 extending into the drill bit 478 from a second or bottom surface toward the first or top surface. The opening 479 is configured or sized and shaped to receive the first shaft 474 of the rotating member 470.

The drive member 430 may also include a sleeve 480 that couples to the second end of the housing 432. The sleeve 480 may have a first end 482 and a second end 484. The sleeve 480 may also include a channel 486 extending through the sleeve 480 from the first end 482 to the second end 484. The channel 486 is configured or sized and shaped to receive a portion of the drive shaft 490. The drive shaft 490 includes a shaft portion 492 and a second worm gear 500. The drive shaft 490 may also include a first protrusion 494 extending from the shaft portion 492 near the first end of the shaft portion 492. The shaft portion 492 also includes a second protrusion 496 extending from the shaft portion 492 between the first end and the first protrusion 494. The first protrusion 494 may be, for example, spaced apart from the second protrusion 496. The shaft portion 492 may also include a recessed shaft portion 498 positioned between the second protrusion 496 and the second worm gear 500. The diameter of the recessed shaft portion 498 may be, for example, smaller than the diameter of the shaft portion 492, the protrusions 494, 496, and the second worm gear 500. The second worm gear 500 may be, for example, positioned near the first end of the drive shaft 490. The drive shaft 490 may also include an alignment tab 502 at the second end of the drive shaft 490. In addition, the alignment pin 504 extends from the alignment tab 502 to the second end. The alignment tab 502 may be for example, flat or planar, while the alignment pin 504 is round or cylindrical.

An instrument 100 with a left sided drive member 430 is shown in FIGS. 1 and 44-47. The left sided drive member 430 may include a left sided housing 510. The left sided housing 510 is similar to the right sided housing 432, however, the left sided housing 510 has a protrusion 512 on the left side of the housing 510, while the right sided housing 432 has a protrusion 442 on the right side of the housing 432. With the exception of the side the protrusion 512 is positioned on in the housing 510, the remaining portions of the left sided drive member 430 are the same as the right sided drive member 430 and will not be described again here for brevity’s sake.

A surgical method for using the instruments 100 may include obtaining an instrument 100 and a tibial cutting guide 110 after a tibial resection cut has been performed as part of a total ankle resection (TAR) procedure. The tray 112 may be positioned such that the top surface of the tray 112 abuts the resected surface of the distal tibia. Next, pins may be inserted through openings 128 in the tibial coupling element 122 to couple the tibial cutting guide 110 with an anterior portion of the tibia. The instrument 100 may also be coupled to the tibial cutting guide 110 prior to or after the tibial cutting guide 110 is secured to the tibia. When the instrument 100 is secured to the cutting guide 110, the drill bit 478 is positioned below one of the drill openings 114, 116. Then, the shaft 146 of the coupling member 140 may be coupled with a powered handle or drill. Next, an upward force is applied to at least one of the coupling portion 130, alignment assembly 300, and driving member 430 such that the tip of the drill bit 478 contacts the resected surface of the distal tibia. The force may be applied by the surgeon or using an additional instrument, for example, a distractor that engages the housing 432, 510. Alternatively, the force may be applied by applying force to the first alignment bar 302 and the second alignment bar 340 to translate the housing 432, 510 and thus, move the drill bit 478 into the resected surface of the distal tibia. Once the drill bit 478 contacts the resected surface, the powered handle or drill (not shown) may be actuated to rotate the first drive member 230, which in turn rotates the second drive member 250, the drive shaft 490, and the first worm gear 464 to initiate rotation of the drill bit 478. As the drill bit 478 is rotated and force is applied, the drill bit 478 may pass through the drill openings 114, 116 of the tibial tray 112 to drill the openings in the tibia for receiving a tibial implant. After the openings are drilled into the tibia, the instrument 100 and tibial cutting guide 110 may be decoupled from each other and the tibia. Finally, the TAR procedure may proceed.

As may be recognized by those of ordinary skill in the art based on the teachings herein, numerous changes and modifications may be made to the above-described and other embodiments of the present disclosure without departing from the scope of the disclosure. The components of the implants as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implants may include more or fewer components or features than the embodiments as described and illustrated herein. Accordingly, this detailed description of the currently-preferred embodiments is to be taken as illustrative, as opposed to limiting of the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. An instrument, comprising:
a coupling portion;
an alignment mechanism coupled to the coupling portion; and
a driving member coupled to the alignment mechanism, wherein a drill bit of the driving member is positioned perpendicular to a housing of the driving member.

2. The instrument of claim 1, wherein the coupling portion comprises:
a drill coupling member;
a drive housing positioned adjacent to the drill coupling member; and
an angled block coupled to the drive housing by at least one fastener.

3. The instrument of claim 2, wherein the coupling portion further comprises:
a first drive member positioned within at least a portion of the drive housing and extending through the drive housing into the drill coupling member; and
a second drive member positioned within at least a portion of the angled block and the drive housing.

4. The instrument of claim 3, wherein a first gear of the first drive member rotatably engages a second gear of the second drive member.

5. The instrument of claim 1, wherein the alignment mechanism comprises:
a first alignment bar;
a second alignment bar; and
at least one coupling system moveably connecting the first alignment bar to the second alignment bar.

6. The instrument of claim 5, wherein the at least one coupling system comprises:
a first coupling system positioned on a first side of the alignment mechanism; and
a second coupling system positioned on a second side of the alignment mechanism.

7. The instrument of claim 5, wherein the at least one coupling system comprises:
a rod with a first end and a second end;
a retaining member engaging a first end of the rod;
a spring moveably positioned around the rod; and
a screw extending through the retaining member and into a portion of the first end of the rod.

8. The instrument of claim 1, wherein the driving member comprises:
a housing;
a sleeve positioned adjacent to the housing; and
a drive shaft extending through the sleeve and a portion of the housing.

9. The instrument of claim 8, wherein the driving member further comprises:
a first worm gear positioned within the housing and engaging a portion of the drive shaft;
a rotating member with a first shaft and a second shaft, wherein the first shaft of the rotating member engages an opening of the first worm gear; and
wherein the drill bit engages the second shaft of the rotating member.

10. The instrument of claim 9, wherein the first worm gear engages a second drive gear of the drive shaft.

11. The instrument of claim 10, wherein a second end of the drive shaft engages a second drive member of the coupling portion.

12. The instrument of claim 11, wherein the first worm gear, a second worm gear of the drive shaft, a first gear of a first drive member, and a second gear of the second drive member are rotatably coupled.

13. A surgical method for an arthroplasty procedure, comprising:
obtaining an instrument and a tibial cutting guide;
attaching the tibial cutting guide to a tibia after a tibial resection is complete;
coupling the instrument to the tibial cutting guide;
aligning a drill bit of the instrument with an opening in the tibial cutting guide;
translating the drill bit through the opening to drill into the tibia; and
removing the instrument and the tibial cutting guide after at least one opening is drilled.

14. The surgical method of claim 13, wherein aligning the drill bit of the instrument with the opening in the tibial cutting guide comprises:
selecting a right sided drill for engaging the opening on a right side of the tibial cutting guide; and
selecting a left sided drill for engaging the opening on a left side of the tibial cutting guide.

15. The surgical method of claim 13, further comprising:
coupling a shaft of a coupling member of the instrument to a powered handle.

16. The surgical method of claim 15, further comprising:
actuating the powered handle to rotate the drill bit and drill the tibia.

17. The surgical method of claim 13, wherein translating the drill bit through the opening to drill into the tibia comprises:
applying force to at least a portion of the instrument.

18. The surgical method of claim 13, wherein translating the drill bit through the opening to drill into the tibia comprises:
translating the drill bit in a direction perpendicular to a bottom surface of the tibial cutting guide and into the tibia.

19. The surgical method of claim 13, wherein the instrument comprises:
a coupling portion;
an alignment mechanism coupled to the coupling portion; and
a driving member coupled to the alignment mechanism, wherein a drill bit of the driving member is positioned perpendicular to a housing of the driving member.

20. The surgical method of claim 19, further comprising:
drilling an opening into the tibia, wherein drilling an opening into the tibia comprises:
rotating a drive shaft of the instrument with a first worm gear of the driving member;
rotating a second worm gear of the driving member with the first worm gear; and
rotating a rotating member of the driving member and the drill bit with the second worm gear.
